(19) United States
(12) Patent Application Publication
(10) Pub. No.: US 2024/0268784 A1
(43) Pub. Date: Aug. 15, 2024

(54) ULTRASOUND PROBE

(71) Applicant: SHEMPRIZ.HEALTH LTD, Ra’anana (IL)

(72) Inventors: Serge PRISANT, Paris (FR); Beatrice CHEMLA, Ra‘anana (IL); Avi SZYCHTER, Ra‘anana (IL)

(21) Appl. No.: 18/568,997

(22) PCT Filed: Jun. 9, 2022
(86) PCT No.: PCT/IL2022/050620

§ 371 Date: Dec. 11, 2023

Related U.S. Application Data
(60) Provisional application No. 63/209,412, filed on Jun. 11, 2021.

(51) Int. Cl. A61B 8/08; A61B 8/00; A61B 8/12

(52) U.S. Cl. CPC A61B 8/085; A61B 8/12; A61B 8/4209; A61B 8/4461; A61B 8/5207

(57) ABSTRACT

A method for scanning a pelvic region, including:

stabilizing a probe within a body cavity comprising a vagina or a rectum of a subject;
scanning a volume of a body region by a scanner of the probe without moving the probe relative to the body cavity body cavity, with a scanning angle in a range of at least 50 degrees.

### RELATED APPLICATION/S

This application claims the benefit of priority under 35 USC § 119(e) of U.S. Provisional Patent Application No. 63/209,412 filed 11 Jun. 2021, the contents of which are incorporated herein by reference in their entirety.

### FIELD AND BACKGROUND OF THE INVENTION

The present invention, in some embodiments thereof, relates to scanning of a body lumen, more particularly, but not exclusively, to scanning of a pelvic region.

Infertile women are submitted to an arduous treatment regimen even though they are mostly young healthy women. In the US, Japan and Europe the majority is composed of working women who may additionally face discrimination and setbacks while they are less available for their careers. As part of the process, women have to submit to multiple iterative Transvaginal Ultrasound exams.

Background art includes, U.S. Pat. No. 10,610,193B2, International Patent Application Publication No. WO17168421A2, U.S. Patent Application Publication No. US2019060675AA, U.S. Patent Application Publication No. US2019110738AA, U.S. Patent Application Publication No. US2018344286AA, U.S. Patent Application Publication No. US2017303903AA, U.S. Patent Application Publication No. US2011188716AA, U.S. Patent Application Publication No. US2014088364AA, and U.S. Patent Application Publication No. US2020315444AA.

### SUMMARY OF THE INVENTION

Some examples of some embodiments of the invention are listed below (an embodiment may include features from more than one example and/or fewer than all features of an example):

Example 1. A method for scanning a pelvic region, comprising:
stabilizing a probe within a body cavity comprising a vagina or a rectum of a subject;
scanning a volume of a body region by a scanner of said probe without moving said probe relative to said body cavity, with a scanning angle in a range of at least 50 degrees.
Example 2. A method according to example 1, wherein said scanning comprising scanning said body volume by emitting an ultrasound beam towards said body region volume from said scanner, wherein said scanner comprises at least one ultrasound transducer.
Example 3. A method according to any one of examples 1 or 2, comprising:
processing scan data acquired during said scanning; and
identifying in said processed scan data of said body volume, reproductive tissue and/or changes thereof.
Example 4. A method according to example 3, wherein said processing comprises determining a relation between said scan data to previously acquired scan data or indications thereof, and wherein said identifying comprises identifying said reproductive tissue and/or changes thereof based on said determined relation.
Example 5. A method according to example 4, wherein said determining comprises comparing said scan data to previously acquired scan data or indications thereof, and wherein said identifying comprises identifying said reproductive tissue and/or changes thereof based on said comparison results.
Example 6. A method according to example 3, wherein said reproductive tissue comprises at least one ovary of said subject.
Example 7. A method according to example 6, wherein said reproductive tissue comprises at least one follicle and wherein said identifying comprises identifying changes in at least one parameter of said at least one follicle and/or at least one parameter of said at least one ovary.
Example 8. A method according to example 7, wherein said identified changes in said at least one parameter of said at least one follicle and/or at least one parameter of said ovary indicating a level of stimulation or maturation of said at least one follicle.
Example 9. A method according to any one of examples 7 or 8, wherein said at least one parameter of said at least one follicle comprises at least one of, size, shape, external surface texture and/or position of said at least one follicle.
Example 10. A method according to any one of examples 7 to 9, wherein said at least one parameter of said at least one ovary comprises number of follicles in said at least one ovary, position of said at least one ovary and/or size of said at least one ovary.
Example 11. A method according to any one of the previous examples, wherein said stabilizing comprises stabilizing said scanner of said probe within said body while said subject sits on said probe, and wherein said method comprises moving an upper part of a body of said subject during said sitting, prior to and/or during said scanning.
Example 12. A method according to any one of examples 1 to 10, comprising:
fixing said probe to an external surface located outside said body cavity prior to said stabilizing.
Example 13. A method according to any one of the previous examples, comprising moving said scanner relative to said probe prior to, during and/or following said scanning.
Example 14. A method according to any one of the previous examples, wherein said stabilizing comprising stabilizing a scanner of said probe at a distance smaller than 5 cm from an external cervix orifice or from a vaginal fornix.
Example 15. A method according to any one of the previous examples, wherein said stabilizing comprises reversibly expanding an anchor coupled to said probe within said body cavity.
Example 16. A system for scanning of a body volume, comprising:
an intracavity ultrasound probe having an elongated body shaped and sized to be introduced at least partly into a body cavity of a subject, wherein said probe comprises:
a scanner comprising at least one ultrasound transducer configured to generate an ultrasound beam and to acquire scan data;
a control device in communication with said intracavity ultrasound probe, comprising a memory and a control circuitry, wherein said control circuitry is configured to determine if said scan data comprises information on a target tissue, by determining a relation between said scan data received from said probe and one or more indications stored in said memory.
Example 17. A system according to example 16, wherein said one or more indications comprise a model of said target tissue and/or previously acquired scan data.
Example 18. A system according to any one of examples 16 or 17, wherein said control unit comprises a user interface configured to generate an alert signal if said scan data does not comprises information on said target tissue.
Example 19. A system according to any one of examples 16 to 18, wherein said probe comprises an anchor coupled to said probe body and configured to move between a collapsed state and an expanded state, and to anchor said elongated body within said cavity in said expanded state.
Example 20. A system according to example 19, wherein said anchor surrounds at least partly said at least one ultrasound transducer.
Example 21. A system according to example 19, wherein said anchor surrounds at least partly said elongated body.
Example 22. A system according to any one of examples 19 to 21, wherein said anchor comprises a balloon, and wherein in an expanded state said balloon is filled with a fluid that allows passage of said ultrasound beam towards a wall of said body cavity in contact with said anchor.
Example 23. A system according to any one of examples 19 to 22, wherein said control circuitry signals said at least one ultrasound transducer to generate and emit said ultrasound beam when said anchor is in an expanded state.
Example 24. A system according to any one of examples 19 to 23, wherein said anchor comprises a balloon.
Example 25. A system according to any one of examples 16 to 24, wherein said probe comprises a handle coupled to said elongated body, and at least one actuator in said handle, wherein said control circuitry signals said at least one actuator to move said scanner and/or said at least one ultrasound transducer prior to and/or during the generation of said ultrasound beam.
Example 26. A system according to any one of examples 16 to 24, wherein said control unit, comprises at least one actuator, and wherein said control circuitry signals said at least one actuator to move said scanner and/or said at least one ultrasound transducer prior to and/or during the generation of said ultrasound bean.
Example 27. A system according to any one of examples 25 or 26, wherein said at least one actuator comprises an electric motor.
Example 28. A system according to any one of examples 25 or 26, wherein said at least one actuator comprises a preloaded actuator.
Example 29. A system for scanning of a pelvic region, comprising:
a transvaginal ultrasound probe having an elongated body shaped and sized to be introduced at least partly into a body cavity of a female subject, wherein said probe comprises:
a scanner comprising at least one ultrasound transducer configured to generate an ultrasound beam and to acquire scan data;
an anchor configured to move between an expanded state and a collapsed state, and wherein in said expanded state said anchor anchors said elongated body to walls of said body cavity;
a control device in communication with said transvaginal ultrasound probe, comprising a memory and a control circuitry, wherein said control circuitry signals said at least one ultrasound transducer to generate said ultrasound beam when said anchor is in said expanded state.
Example 30. A system according to example 29, wherein said anchor surrounds at least partially said at least one ultrasound transducer.
Example 31. A system according to any one of examples 29 or 30, wherein said anchor comprises a balloon, and wherein in an expanded state said balloon is filled with a fluid that allows passage of said ultrasound beam towards a wall of said body cavity in contact with said balloon.
Example 32. A method for detecting changes in reproductive system tissue, comprising:
acquiring a first set of ultrasound scan data of said reproductive system tissue and at least one second set of ultrasound scan data of said reproductive system tissue, wherein said first set of scan data and said at least one second set of scan data are acquired at different time points;
comparing said first set of scan data and said at least one second set of scan data;
detecting changes in said reproductive system tissue based on said comparison results.
Example 33. A method according to example 32, wherein said acquiring comprises acquiring said first set of scan data and said at least one second set of scan data from the same location.
Example 34. A method according to any one of examples 32 or 33, wherein said first set of scan data and said at least one second set of scan data comprises two-dimensional images.
Example 35. A method according to any one of examples 32 to 34, comprising diagnosing endometriosis based on said detected changes.
Example 36. A method according to any one of examples 32 to 34, wherein said reproductive system comprises at least one ovary and two or more follicles, and wherein said method comprises determining a stimulation state of said follicles based on said detected changes.
Example 37. A method according to any one of examples 32 to 34, wherein said reproductive system comprises at least one ovary and wherein said method comprises diagnosing or monitoring polycystic ovary syndrome (PCOS) based on said detected changes.
Example 38. A method according to any one of examples 32 to 34, wherein said reproductive system tissue comprises at least one of uterus and cervix, and wherein said method comprises detecting pre-term labor based on said detected changes.
Example 39. A method according to any one of examples 32 to 34, wherein said reproductive system comprises at least one of, at least one ovary, a uterus, a cervix, and wherein said method comprises diagnosing a tumor or monitoring a tumorigenic process based on said detected changes.
Example 40. A method according to any one of examples 32 to 39, wherein said acquiring comprises acquiring said first set of scan data and said at least one second set of scan data from a similar position in a body cavity comprising a vagina or rectum.
Example 41. A method according to any one of examples 32 to 40, wherein said acquiring comprising acquiring said first set of ultrasound scan data and at least one second set of ultrasound scan data from within a body cavity, and wherein said method comprises stabilizing an ultrasound probe within said body cavity during said acquiring.
Example 42. A method according to example 41, wherein said body cavity comprises a vagina.
Example 43. A method for endometrial monitoring, comprising:
scanning a uterus using a transvaginal ultrasound probe positioned within the vagina, without moving said transvaginal ultrasound probe during said scanning;
identifying endometrial lining in said scanned uterus; and
monitoring changes in at least one of, stage, shape and/or size of said identified endometrial lining.
Example 44. A method according to example 43, comprising:
determining if the uterus is ready for embryo transfer based on said monitored changes.
Example 45. A method for follicular monitoring, comprising:
scanning one or two ovaries using a transvaginal ultrasound probe positioned within the vagina, without moving said transvaginal ultrasound probe during said scanning;
identifying two or more follicles in said scanned one or two ovaries; and
monitoring changes in at least one of, number, shape and/or size of said identified two or more follicles.
Example 46. A method according to example 45, comprising:
determining a maturity status of oocytes in said identified one or two follicles based on said monitored changes.
Example 47. A method according to example 45, comprising:
determining a response of said two or more identified follicles to an ovarian stimulation treatment based on said monitored changes.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

As will be appreciated by one skilled in the art, some embodiments of the present invention may be embodied as a system, method or computer program product. Accordingly, some embodiments of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, some embodiments of the present invention may take the form of a computer program product embodied in one or more computer readable medium(s) having computer readable program code embodied thereon. Implementation of the method and/or system of some embodiments of the invention can involve performing and/or completing selected tasks manually, automatically, or a combination thereof. Moreover, according to actual instrumentation and equipment of some embodiments of the method and/or system of the invention, several selected tasks could be implemented by hardware, by software or by firmware and/or by a combination thereof, e.g., using an operating system.

For example, hardware for performing selected tasks according to some embodiments of the invention could be implemented as a chip or a circuit. As software, selected tasks according to some embodiments of the invention could be implemented as a plurality of software instructions being executed by a computer using any suitable operating system. In an exemplary embodiment of the invention, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized for some embodiments of the invention. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present invention may be described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer, and may not be feasible or practical for performing purely manually, by a human expert. A human expert who wanted to manually perform similar tasks, such as, for example determining a position and/or state of organs based on ultrasound imaging, might be expected to use completely different methods, e.g., making use of expert knowledge and/or the pattern recognition capabilities of the human brain, which would be vastly more efficient than manually going through the steps of the methods described herein.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a flow chart of a general process for determining tissue state, according to some exemplary embodiments of the invention;

FIGS. 2A and 2B are a schematic illustrations showing ultrasound scanning of a body volume, according to some exemplary embodiments of the invention;

FIG. 2C is a flow chart of a process including system and patient interactions, according to some exemplary embodiments of the invention;

FIGS. 3A-3C are schematic block diagrams of a probe, according to some exemplary embodiments of the invention;

FIG. 3D is a schematic block diagram of a probe coupled to a control unit, according to some exemplary embodiments of the invention;

FIGS. 4A-4C are schematic block diagrams of a system, according to some exemplary embodiments of the invention;

FIG. 5A is a flow chart of a process for using a probe by a patient, according to some exemplary embodiments of the invention;

FIGS. 5B and 5C are flow charts of a scanning describing system activities, according to some exemplary embodiments of the invention;

FIG. 6A is a flow chart of a general procedure for processing of scan data, according to some exemplary embodiments of the invention;

FIG. 6B is a flow chart of a detailed procedure for identifying changes in target tissue, according to some exemplary embodiments of the invention;

FIG. 6C is a flow chart of an additional detailed procedure for identifying changes in target tissue, according to some exemplary embodiments of the invention;

FIG. 7A is a schematic illustration of a generated point cloud, according to some exemplary embodiments of the invention;

FIGS. 7B-7C are schematic illustrations of planes, according to some exemplary embodiments of the invention;

FIGS. 8A-8H are schematic illustrations of a probe, according to some exemplary embodiments of the invention;

FIGS. 9A-9F are schematic illustrations of a probe, according to some additional exemplary embodiments of the invention;

FIGS. 10A-10D are schematic illustrations of a probe positioned within the vagina and during scanning, according to some exemplary embodiments of the invention;

FIGS. 11A-11J are additional schematic illustrations of a probe positioned within the vagina and during scanning, according to some additional exemplary embodiments of the invention;

FIG. 12A is an illustration showing different applications of the system, according to some exemplary embodiments of the invention;

FIG. 12B is an illustration showing flow of information within the system, according to some exemplary embodiments of the invention;

FIG. 12C is an illustration showing interaction between a female subject and the system. according to some exemplary embodiments of the invention;

FIGS. 13A-13D are schematic illustrations showing movement of a body of a subject while an ultrasound probe is stabilized within the subject body, according to some exemplary embodiments of the invention; and

FIGS. 13E-13G are schematic illustrations showing a position of the ovaries relative to a stabilized probe during and/or following body movements, according to some exemplary embodiments of the invention.

### DESCRIPTION OF SPECIFIC EMBODIMENTS OF THE INVENTION

The present invention, in some embodiments thereof, relates to scanning of a body lumen, more particularly, but not exclusively, to scanning of a pelvic region.

### Overview

An aspect of some embodiments of the invention relates to scanning a pelvic region by positioning of a scanning probe, for example an ultrasound probe, at a specific location during the scanning. In some embodiments, the specific location is within a vagina of a female subject. In some embodiments, an axial position and/or an angular position of the probe is fixed during the scanning. As used herein, stabilizing or placing a probe in a specific location and/or in a fixed position during scanning means moving or rotating the probe in less than 5 cm, for example less than 3 cm, less than 1 cm, less than 5 mm, less than 1 mm or any intermediate, smaller or larger distance in all directions during the scanning.

According to some embodiments, the probe is stabilized within the body cavity, for example within the vagina or rectum during a scanning process of a body region volume, for example a volume of the pelvic region. In some embodiments, when the probe is stabilized, the probe is stationary from insertion of the probe at least partly into the body cavity and until retrieval of the probe out from the body cavity, and during the scanning process, and optionally during the whole scanning session. In some embodiments, the probe is stabilized at a distance smaller than 5 cm, for example at a distance smaller than 3 cm, smaller than 1 cm, smaller than 0.5 cm, or any intermediate, smaller or larger distance from an external cervix orifice or from a vaginal fornix.

According to some embodiments, the probe is stabilized using an anchor, for example an expandable anchor functionally coupled to the probe body. Alternatively or additionally, the probe is stabilized by coupling of the probe to an external surface positioned outside the body. In some embodiments, the external surface comprises a ball, a chair, a wall or a floor. In some embodiments, the external surface is a surface of an applicator, configured to hold and stabilize the probe from outside the body during scanning. Optionally, the applicator is configured to introduce, for example controllably introduce the probe into the body cavity.

A potential advantage of stabilizing the probe during scanning may be to allow scanning of a body volume by a non-expert person, and/or to allow self-examination without a need of an expert help to move the probe within the body cavity.

According to some embodiments, the scanning comprises scanning an overall sector of a selected body volume with a scanning angle, for example a subtended angle of at least 45degrees, for example an overall sector having a subtended angle of at least 50 degrees, of at least 90 degrees, of at least 180, of at least 270 degrees, or any intermediate, smaller or larger value, while the probe is positioned in the specific location. In some embodiments, the scanning comprises scanning an overall sector using a scanning angle, for example a subtended angle in a range between 45 degrees and 360 degrees, for example a subtended angle in a range between 45 degrees and 100 degrees, a subtended angle in a range between 90 degrees and 200 degrees, or any intermediate, smaller or larger range of values.

According to some embodiments, during a scanning session, a body volume is scanned, for example from side-to-side to reach an overall scanning angle of at least 30 degrees, for example at least 50 degrees, at least 90 degrees, at least 180 degrees, or any intermediate, smaller or larger scanning angle. In some embodiments, a scanning session is a session that initiates when the probe is introduced into a body cavity, until it is removed from the body cavity.

According to some embodiments, a scanning beam is moved between several angular positions relative to a long axis of the scanning probe, to scan at least two, for example a plurality of sub-sectors of the overall sector, while the probe is position at the specific location during movement of the scanning beam. In some embodiments, each sub-sector has a subtending angle in a range between 0.5 degrees and 20 degrees, for example a subtending angle in a range between 0.5 degrees and 5 degrees, between 1 degrees and 10 degrees, between 3 degrees and 20 degrees, or any intermediate, smaller or larger range of values.

According to some exemplary embodiments, at least one energy emitting transducer emitting the scanning beam moves intermittently between the several angular positions prior to scanning. Alternatively, the at least one energy emitting transducer moves continuously between the several angular positions.

According to some exemplary embodiments, scanned images of the sub-sectors are used to identify tissue, for example organs in the pelvic region. In some embodiments, the tissue comprises at least one of, Uterus, at least one Ovary, Follicle, and Bladder and or prostate. In some embodiments, the scanned images are pre analyzed, for example to identify a nature of a tissue/organ, optionally using neural network. Optionally, a region of interest is analyzed along two or more orthogonal planes, and constitute a set of training data to teach the network.

In some embodiments, scanned images of the sub-sectors are used to generate a 3D image of the overall sector.

According to some exemplary embodiments, the scanned images of the sub-sectors are used to detect a change in at least one parameter of the tissue compared to stored information, for example shape, size, and granularity of the tissue. In some embodiments, the stored information comprises previously scanned images or indications of values or range of values of the at least one parameter.

An aspect of some embodiments relates to transmitting a sideways ultrasound scanning beam from an ultrasound probe within a body cavity. In some embodiments, the ultrasound scanning beam is transmitted sideways at an angle of up to 180 degrees relative to a longitudinal axis of the body cavity, for example a vagina. In some embodiments, at least one ultrasound transmitter transmitting the sideways ultrasound scanning beam is a sideways facing ultrasound transducer. Alternatively or additionally, a window or an aperture of the probe through which the ultrasound scanning beam passes, is a sideways facing window.

An aspect of some embodiments relates to a scanning system comprising an ultrasound probe, for example a transvaginal ultrasound probe, that delivers one or more indications regarding a scanning procedure to a patient while the ultrasound probe is positioned within a body cavity of the patient. In some embodiments, the one or more indications comprise at least one indication regarding a reminder to initiate scanning, scanning initiation and/or stopping of the scanning. Alternatively or additionally, the one or more indications comprise at least one indication regarding at least one of, position of the ultrasound probe within a body cavity, for example a vagina, stabilization of the probe within a specific location within the body cavity and contact between the ultrasound probe, for example a scanning portion of the ultrasound probe, and a wall of the body cavity. Alternatively or additionally, the one or more indications comprise a reminder to start a scanning session, for example a time period in which a probe is placed within the body cavity and performs scanning. According to some exemplary embodiments, the one or more indications comprise a human detectable indication, for example an audio indication, a visual indication, a sensory indication, and a tactile indication. In some embodiments, the one or more indications are delivered by the probe, for example by a user interface located in the probe handle. Alternatively or additionally, the one or more indications are delivered by a device functionally coupled to the probe. In some embodiments, the device coupled to the probe comprises at least one of a, wearable device for example a smartwatch or a smart band, a virtual personal assistant device, a cellular phone device or any other device functionally coupled to the probe via wires or via wireless communication.

A potential advantage of receiving reminders regarding a scanning procedure may be to assist a patient to self-operate a scanning device positioned within the patient body with minimal knowledge and experience.

An aspect of some embodiments relates to stabilizing a position of an ultrasound probe within a body cavity, for example the vagina or rectum, using at least one anchor. In some embodiments, the anchor is configured to move between a collapsed state, for example during the insertion of the probe into the body cavity, to an expanded state, for example when the probe is stabilized within the body cavity.

According to some exemplary embodiments, the at least one anchor is configured to prevent lateral and/or axial movements of the probe within the cavity, when the at least one anchor is in the expanded state. In some embodiments, the at least one anchor comprises a balloon. In some embodiments, the balloon is filled with a liquid, in order to expand. In some embodiments, in an expanded state, the anchor is in contact with the body cavity wall. In some embodiments, the anchor surrounds, at least partly the at least one ultrasound transducer of the probe. Optionally, when using a balloon filled with liquid, the balloon is also used to maintain contact, for example ultrasound contact between the at least one ultrasound transducer of the probe and the body cavity wall.

An aspect of some embodiments relates to scanning of a body volume using movements of the body, for example a portion of the body. In some embodiments, movements of the body change a position and/or orientation of tissue within the body volume relative to a scanner, for example a scanner that comprises at least one ultrasound transducer and/or a scanner that comprises an optics assembly. In some embodiments, the scanner is positioned outside the body. Alternatively the scanner is positioned within a body cavity.

According to some embodiments, a probe comprising the scanner is fixedly coupled to an external surface stabilizing the probe position outside the body or within the body. In some embodiments, the probe is fixedly coupled to a chair or a ball that allows a user to sit in the probe.

According to some exemplary embodiments, the system is a pelvis imaging system aiming at recording and processing images acquired by an ultrasonographic head containing an ultrasound transducer. In some embodiments, the transducer comprising an array of transducer elements. In some embodiments, the system is configured and able to process at least a part of the images acquired by the probe for calculating transformations to a “reference image” repository relative to the initial position of pelvis organ such as ovaries, ovarian follicles, uterus, fallopian tubes, kidneys, digestive system, with at least a part of the images acquired being recorded with a view to visualizing representations thereof on individual images of each structure.

According to some exemplary embodiments, an ultrasound probe comprises a transducer head (optionally associated to a moving system) housed inside an adult novelty type designed body and adapted to mobilize a transducer array. In some embodiments, the ultrasound probe can be further associated with an expandable system to ensure perfect application of the probe to the area of interest and a controller system to further promote the autonomous acquisition of images. In some embodiments, the system is associated with a software to promote human machine interaction (HMI), collect data and integrate various features from the adult novelty lines and the entertainment universe. Optionally, a dedicated and secure application can be associated to the system for the promotion of workflow fluidity. In some embodiments, the system is used in reproductive health, women health, urogenital aging and urogenital cancer screening.

According to some exemplary embodiments, the imaging system can further include at least one motor for autonomous image acquisition, the transducer head and motor included in a probe that is shaped to be easily inserted or positioned, connected to an energy source, constituting a moving system. In some embodiments, the system can be included into a support and contact module (SCM).

According to some exemplary embodiments, the probe is a manually inserted/positioned probe.

According to some exemplary embodiments, the imaging system together with the moving system allow a full volume scan solution, without any intervention of the woman, letting the device acquire images from the pelvis volume in an automated fashion.

According to some exemplary embodiments, the moving system is equipped with a coding system of position of the axis(es), in order to know in real time, the absolute position of the acquisition module, and by then, the full device is capable to locate precisely in the 3d dimension of the body, the position of all the follicles and the ovaries, and/or all the medical tissues that are relevant for the device.

According to some exemplary embodiments, in the moving system not only the mechanical rotation is monitored in real time, but also the system is capable at any time, to reach this position, thanks to a coding system, which is referenced by an absolute zero position.

According to some exemplary embodiments, in the moving system not only the position is monitored in absolute, but also that the system is capable to address at any time during an acquisition period (between the introductions by the patient, up to the take-out) the absolute position that would have been stored in the memory.

According to some exemplary embodiments, the imaging system will make quickly a 1st scan of the full volume. The term “quickly” is evaluated as less to what the state of the art considered as acceptable for a patient not to breathe and not to move. As an example, quickly can be less than 5 seconds, for example less than 4 seconds, less than 3 seconds, less than 2 seconds or any intermediate, smaller or larger time period.

According to some exemplary embodiments, the imaging system will analyze the acquired volume, using algorithms, in order to determine preferred planes where the medical tissues to analyze are located. In some embodiments, each plane is optionally analyzed, using a segmentation algorithm, and/or a neural network, for example to determine a quality and/or a relevancy of each plane.

According to some exemplary embodiments, the imaging system not only will find out the location in the patient's context, but also will make a 2nd scan, but focusing only on the best planes for measurements and analysis of the key interest points (KPI). In some embodiments, the 2nd scan will be done in a “quick” timing, corresponding at the state of the medical art.

According to some exemplary embodiments, the imaging system will perform, as much scans as required, to reach to the best planes in order to focus the acquisition with the full resolution. In some embodiments, between 2 scans, the patient will be allowed to breathe and move, but with the system in position into the human body.

According to some exemplary embodiments, the imaging system includes an interface that will inform the patient when to move and when not to move. In some embodiments, this interface can be, but not only, a cell phone, or alternatively, any screen or connection device. Optionally the signal can be, but not limited to visual, sound, music, sensorial.

According to some exemplary embodiments, the imaging system is introduced in the cavity of the patient, or alternatively, the patient can sit on the device.

According to some exemplary embodiments, when the imaging system is inserted or positioned on the abdomen or if the customer sits on it, then the operating position becomes self-evident.

According to some exemplary embodiments, when the customer is sitting on the device. movements in order to acquire images could be predetermined and communicated to the customer via but not limited to an instruction booklet, informative session with a professional or a company representative, a movie, or any other form of communicating the instructions

According to some exemplary embodiments, the imaging system acquires the full volume while the customer follows a predetermined set of movements.

According to some exemplary embodiments, the imaging system acquires the full volume while the customer is not moving. In some embodiments, movement of the imaging system can be done by, but not limited to, a motor, such as a stepper motor or a brushless motor or a dc motor.

According to some exemplary embodiments, the imaging system acquires the full volume while the user is not moving. In some embodiments, the imaging system mobilization can be obtained via, but not limited to, electronic means.

According to some exemplary embodiments, the imaging system acquires the full volume while the customer is not moving. In some embodiments, movement of the imaging system can be done by, but not limited to, a mechanical component, such as a small inertial system that will store the energy to restitute it.

According to some exemplary embodiments, a solution that will avoid an electronized motor (such as, but not limited to, a stepper motor,) or any other solution, allows to avoid EMC and acoustic noise.

According to some exemplary embodiments, a mechanical solution, can be charged in energy thanks to any motor, such as a stepper motor.

According to some exemplary embodiments, a mechanical solution is used, in order to acquire the ultrasound acquisition, with the motor not working (during the time the energy is released).

According to some exemplary embodiments, the imaging system includes a controller system configured to execute programmed instructions.

According to some exemplary embodiments, the imaging system includes a calculator recording and processing the images acquired by the ultrasonographic head. In some embodiments, the calculator also records and processes images required by the probe in order to calculate transformations to a “reference image” repository relating to an initial position of a structure, with at least a part of the images acquired during successive acquisitions being recorded with a view to visualizing representations thereof on individual images of each structure.

According to some exemplary embodiments, the imaging system comprises a refreshing frequency of the images acquired by the ultrasonographic probe being at least 3 images per second.

According to some exemplary embodiments, the imaging system uses the ultrasonographic probe to give a three-dimensional image.

According to some exemplary embodiments, a step of processing the acquired images further implements an image resetting algorithm based on the local optimization of a measure of the similarity.

According to some exemplary embodiments, the imaging system further comprises validating recalculated acquired image.

According to some exemplary embodiments, the imaging system, further comprising integrating other images previously or subsequently acquired to superimpose these on the reference image and previous images of the same structure.

According to some exemplary embodiments, a target section comprises the ovaries, the uterus with endometrium and the fallopian tubes.

According to some exemplary embodiments, the system is configured to monitor reproductive health.

According to some exemplary embodiments, the system is configured to detect early in the reproductive life, women health conditions such as but not limited to, endometriosis, PCOS, adenomyosis, cysts.

According to some exemplary embodiments, the system is configured to monitor women health conditions such as but not limited to, endometriosis, PCOS, adenomyosis, cysts.

According to some exemplary embodiments, the system is configured to monitor when to conceive or not conceive according to the tracking of ovulatory cycles.

According to some exemplary embodiments, the system is configured to provide biofeedback on the pelvic organs situation.

According to some exemplary embodiments, the system is configured to evaluate aging of reproductive and urogenital organs.

According to some exemplary embodiments, the system is configured to screen ovarian parenchyma, uterus tissue bladder or prostate for early cancer detection.

Intravaginal ultrasound probes have been in use for many years for imaging the vagina, including into the vagina (IVT probes) or rectum (ICT probes) to image the cervix, uterus, and/or prostate

According to some embodiments, the system is a comprehensive system, allowing a patient to self-perform a vaginal or rectal examination, without any particular skills. According to some embodiments, the system is a comprehensive system, allowing a person to perform for someone else a vaginal or rectal examination, without any particular skills. In some embodiments, the system is based on an ultrasound technology, can be based on any kind of ultrasounds technology, for instance, and without any limitation, PZT array transducers, CMUT technology.

According to some embodiments, a probe of the system is shaped and sized to allow a head of the probe to reach a fornix or a prostate, and to be placed in contact with the mucosa. In some embodiments, the probe is physically guided up to the fornix or the cervix. In some embodiments, the probe is configured to allow repeatable positioning and contacting with the mucosa.

According to some embodiments, the probe is managed by a dedicated software program that checks continuously or intermittently a position of the uterus, and the ovaries, relative to a contact point between the probe and the mucosa

According to some exemplary embodiments, the probe is functionally coupled to a motor. In some embodiments, the motor is located within the probe, for example within the probe handle. Alternatively the motor is located in a removable part of the handle, within a cable up to a human-machine interface (HMI), for example a cellular phone or within a remote docking station.

According to some embodiments, the motor allows the probe to perform a full volume scan in a single sweep. In some embodiments, the system aligns the probe with each ovary, without any intervention of the patient. In some embodiments, the system is designed for moving the ultrasound plane of acquisition indistinctly from left to right, or the opposite, while knowing a position of the array of transducers at any time. Optionally, the system is capable to look for a specific angle, and make an image acquisition at this specific angle

According to some embodiments, the system is designed for acquiring any angle between +180/−180 deg, or any other subset of this interval

According to some embodiments, the probe is managed by a software to allow generation and comparison of 3D images, for example between two scanning sessions.

In some embodiments, positioning a probe in a specific location in each scanning session, allows the system to compare acquired data between the two scanning sessions

According to some exemplary embodiments, the system identifies in real time a position of the uterus and the ovaries, or any other anatomical landmark, to allow comparison between scan data, for example images acquired in two or more scanning sessions (same day or different days) and optionally to precisely overlap 3D volume.

According to some exemplary embodiments, the system is designed so that, if for any reason, a contact with the mucosa is not exactly as in another vagina insertion, then the system knows how to recalculate the difference in position, for example to guarantee the overlap.

According to some embodiments, the system is designed to contain a small amount of gel, to guarantee the contact of the probe with the mucosa, while adding enough gel to facilitate the ultrasound continuity from the transducers array up to the ovaries,

According to some embodiments, the system is equipped with a solution of expandable envelop, filled with gel, in order to compensate different touch point with the mucosa.

According to some embodiments, the probe comprises a storage container filled with an echographic solution, for instance but not limited to, an echographic, for example an ultrasound compatible gel and/or fluid. In some embodiments, this solution is released from the storage container, for example when the probe is stabilized within the vagina. In some embodiments, the solution is released between the probe and the vagina wall. Alternatively, the solution is released to a balloon, at least partly surrounding at least one ultrasound transducer of the probe. Optionally, the balloon is inflated by introducing the solution into the balloon, for example using a syringe, optionally from outside the vagina.

According to some embodiments, the system is designed to handle huge amount of data, without any particular system-external solution, for example by adding a computing unit, and/or by embedding part or the computing unit in a cloud, for example a secured cloud.

According to some embodiments, the system is built in such a way, that each 2d plane can be acquired, in an electronic scanning of the full 2D field of view. According to some embodiments, the system allows to select a Region of Interest (ROI) for each woman, and to optionally perform a specific scanning of the ROI.

According to some embodiments, the system perform pre-processing process by the Point of care (the home of the patient). In some embodiments, the preprocessing comprises a compression of the images directly in the point of care, before sending images into the cloud.

According to some embodiments, the system is configured to associate physical parameters of the acquisition, for example acquisition coordinates with a specific patient and/or with a specific Point of Care.

According to some embodiments, a probe and acquisition parameters are personalized according to a physiology of each patient.

According to some embodiments, a probe is associated with a single patient, for example to avoid cross contamination. In some embodiments, the probe is coded with a personal code acquired by the user. In some embodiments, the code allows a specific number of scans or allows activation of the probe within a predetermined time. In some embodiments, the code allows to personalize a probe for a specific user, for example by associating the probe with a specific user profile.

The System is designed for having the probe being separated to the rest of the system, that can circulate between patients. This represents a progress to the state of the art, since neither the probe, nor the ultrasound data acquisition platform (the electronic hardware/software managing the probe) can leave the doctors laboratory.

According to some embodiments, the system is designed in such a way that any digital device of the user will be connectable to the system, for hosting the HMI that will guide them during the acquisition.

According to some embodiments, the system comprises a cloud environment, for example, to allow exchanges of data with the doctors, and/or users. In some embodiments, the cloud environment stores images acquired by the point of care, and user. In some embodiments, the cloud is HIPAA compatible under GDPR privacy regulations. Optionally, the cloud and other devices coupled to the system are equipped with cybersecurity protection.

According to some embodiments, the system is configured to organize the collected data, for example to allow to track the evolution of the 3D acquisitions with the time, for each patient, and to optionally generate a transversal data base, between many patients.

According to some embodiments, processing of data is performed by algorithms, for example artificial intelligence algorithms in the cloud and optionally not in the Point of Care.

According to some embodiments, the system receives input from the user and uses the input for improvement of the system software, and/or algorithms.

According to some embodiments, using a cloud or a central server, allows experts, for example physicians treating a specific patient to access all relevant data directly.

According to some embodiments, the system comprises an expert user interface to allow the expert to manage directly the device and the acquisition, optionally without any contact with the patient.

According to some embodiments, the system is configured to process a plurality of 2D images acquired by the system by the Point of care, to create a volumetric model, for example point of cloud, xyz format, 3mf, that is used as an input for image processing and/or for the AI algorithms.

According to some embodiments, the system generates indications and provides information that can be visualized using computers, tablets, and/or AR/VR solutions.

According to some embodiments, processing of the scan data allows, for example, to identify target tissue or changes thereof, and/or to identify that the probe is located at a target position within the body cavity.

According to some embodiments, the scanning system identifies, for example automatically identifies that the scan data includes tissue selected for scanning. In some embodiments, the scanning system identifies the tissue, for example by comparing the scan data to a previously acquired scan data. In some embodiments, the system identifies in the scan data landmarks, for example specific anatomical landmarks in the scanned volume, for example at least one ovary, cervix, uterus, follicles. In some embodiments, the system uses the identified landmarks to compare between the scan data and previously acquired scan data, and to determine if the scanned volume is similar to the previously acquired scanned volume.

Alternatively or additionally, the system compares the scan data to a model stored in a memory associated with system. In some embodiments, the model comprises indications regarding at least one of a shape, size, texture, location, positon of tissue in the scanned volume. In some embodiments, the system determines if the acquired scan data includes scan data of target tissue based on the comparison between acquired scan data and the model.

According to some embodiments, the system detects tissue in a scanned volume, and/or determines changes in the detected tissue by matching between two sets of scanned data acquired in different time points. In some embodiments, the system performs matching of pixels between the two sets of scan data, followed by segmentation of tissue, for example at least one organ, in the two sets of scan data, for example to determine changes in the segmented portions of the tissue between the two sets of scan data.

According to some embodiments, the system is used for follicle monitoring. In some embodiments, two or more sets of scan data acquired at different time points are matched, for example aligned. Optionally, one or more sets are deformed, and/or rotated prior to and/or during the alignment process. In some embodiments, at least one vary or the two ovaries in the sets of scan data are segmented, for example to identify a specific ovary in two or more of the scan data sets. Optionally, an alignment of the specific ovary is performed in 3D. In some embodiments, the system detects changes in the aligned ovary over time between the different sets of scan data. In some embodiments, the changes include changes in at least one of, shape, size, position, surface texture of at least one follicle of the ovary over time.

According to some embodiments, the system is used to at least one of, diagnose, monitor and detect physiological and/or pathological conditions of tissue, for example tissue of the reproductive system. In some embodiments, the system compares a first set of scan data and a second set of scan data of tissue of the reproductive system. In some embodiments, the second set of scan data is acquired at a different time point from the first set of scan data, for example after an hour, after a day, after a month, after a year, or any intermediate, smaller or larger time period. In some embodiments, the system detects, optionally automatically, changes in the reproductive system tissue based on the comparison results.

In some embodiments, the system is used to diagnose endometriosis based on the detected changes. In some embodiments, for example when the reproductive tissue comprises at least one ovary and two or more follicles, the system is used to determine a stimulation state of the follicles based, for example, on the detected changes. In some embodiments, for example when the reproductive system tissue comprises at least one ovary, the system is used for diagnosing or monitoring polycystic ovary syndrome (PCOS) based, for example, on the detected changes. In some embodiments, for example when the reproductive system tissue comprises at least one uterus and cervix, the system detects pre-term labor, for example, based on the detected changes. In some embodiments, for example when the reproductive system tissue comprises at least one of, at least one ovary, a uterus, and a cervix, the system is used for diagnosing a tumor or monitoring a tumorigenic process, for example, based on said detected changes.

According to some embodiments, the system is used for endometrial monitoring. In some embodiments, a transvaginal probe is used to scan the uterus, without moving the probe within the vagina during the scanning. In some embodiments, endometrial lining is identified in a scanned data of the uterus, and changes in at least one of, stage, shape and/or size of the identified endometrial lining are monitored over time, for example by repeating the scanning of the uterus. In some embodiments, the system determines if the uterus is ready for embryo transfer based on the monitored changes.

According to some embodiments, the system is used for follicular monitoring, for example for follicular monitoring during an in-vitro fertilization process. In some embodiments, one or two ovaries are scanned using a transvaginal probe without moving the probe within the vagina during the scanning. In some embodiments, two or more follicles are identified in the scanned ovaries. In some embodiments, the scanning and identifying processes ae repeated in one or more additional scanning sessions. In some embodiments, changes in at least one of, number, shape and/or size of the identified follicles are monitored over time. In some embodiments, a maturity status of oocytes in the identified follicles is determined based on the monitored changes. Alternatively or additionally, a response of the two or more identified follicles to an ovarian stimulation treatment is determined based on the monitored changes, In some embodiments, the system is used to monitor a maturity status of one or more oocytes during a natural maturation process of the one or more oocytes, not related to an IVF treatment.

In some embodiments, the system can be operated by an expert. In some embodiments, the expert navigates and/or extracts the probe from a body cavity using a user interface of a control unit functionally coupled to the probe, for example using a joystick

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

### Exemplary General Process for Ultrasound Scanning

According to some exemplary embodiments, an ultrasound probe, for example a transvaginal probe is introduced into the vagina. In some embodiments, a scanning portion of the probe is positioned at a specific location within the vagina. Optionally, the scanning portion of the probe is placed in contact with the vagina wall at the specific location. In some embodiments, the probe transmits an ultrasound beam, for example a scanning beam, at different directions, to capture an image of the pelvic region, while keeping the probe in the specific position. In some embodiments, the captured image is used to identify tissue for example at least one organ and/or changes thereof, in the pelvic region.

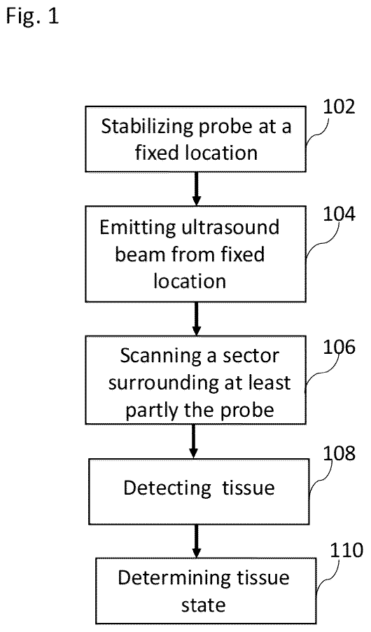

Reference is now made to FIG. 1, depicting a process for identifying and determining a state of tissue using ultrasound energy, according to some exemplary embodiments of the invention.

According to some exemplary embodiments, an ultrasound probe is stabilized at a specific location within a body cavity, for example an elongated body cavity, at block 102. In some embodiments, the body cavity comprises a vagina and/or a rectum. In some embodiments, stabilizing the probe at block 102 comprises ensuring ultrasound contact between the probe, for example a scanning portion of the probe, and a wall of the body cavity at the specific location.

According to some exemplary embodiments, an ultrasound beam, for example a scanning beam, is emitted from the ultrasound probe, at block 104. In some embodiments, the ultrasound beam is emitted while the probe, for example a scanning portion of the probe, is at the specific location. In some embodiments, the ultrasound beam is emitted at different angles relative to a longitudinal axis of the probe, for example towards different sub-sectors of a body volume. In some embodiments, the ultrasound beam is emitted sideways, for example at an angle in a range between 10 degrees and 180 degrees relative to the longitudinal axis of the probe.

According to some exemplary embodiments, at block 104, during the scanning, the ultrasound beams moves between two or more angular positions, each is directed towards a different sub-sector of the body volume. In some embodiments, at least some of the sub-sectors are spaced-apart. Alternatively or additionally, at least some of the sub-sectors overlap.

According to some exemplary embodiments, an overall sector of the body volume surrounding at least party the probe, is scanned at block 106. In some embodiments, the overall sector is scanned by combining scan data received from the different sub-sectors. Optionally, the sub-sectors scan data is combined to generate a three dimensional (3D) image of the body volume.

According to some exemplary embodiments, tissue, for example organs, within the scanned overall sector is detected, at block 108. In some embodiments, detecting tissue comprises identifying that a tissue detected in a scanned data from a sub-sector is similar to tissue detected in a previous scan. Alternatively or additionally, detecting tissue comprises identifying a specific tissue, for example a specific organ. In some embodiments, tissue is identified based on a relation between the identified tissue and one or more anatomical locations. Alternatively or additionally, the tissue is identified based on a relation between the tissue and at least one other tissue, for example at least one other organ, detected in a sub-sector or in the overall sector.

According to some exemplary embodiments, a state of the detected tissue is determined at block 110. In some embodiments, determining a state of the tissue comprises determining values of at least one parameter of the tissue, for example shape, size, location, and/or granularity of the tissue. Alternatively or additionally, determining a state of the tissue determining changes in the at least one parameter compared to stored indication, for example compared to previously scanned data of the tissue. In some embodiments, the determined state indicates a physiological condition or a pathological condition of the tissue.

### Exemplary Sub-Sectors Scanning

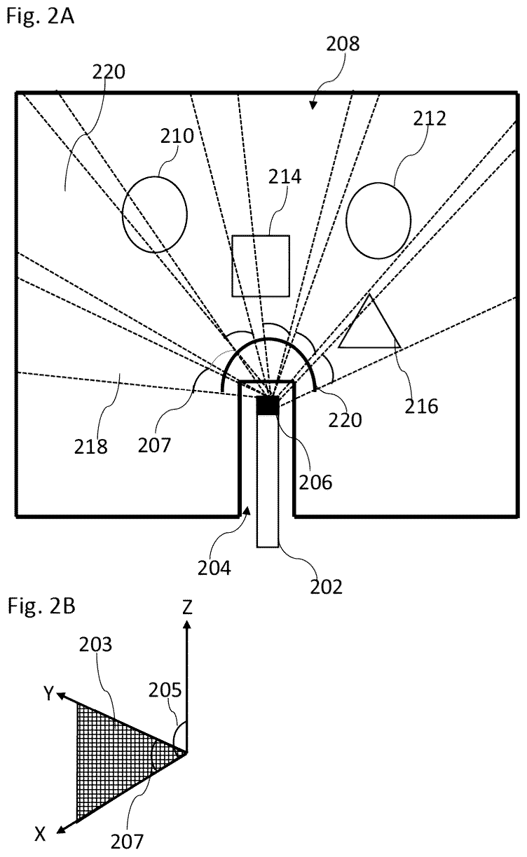

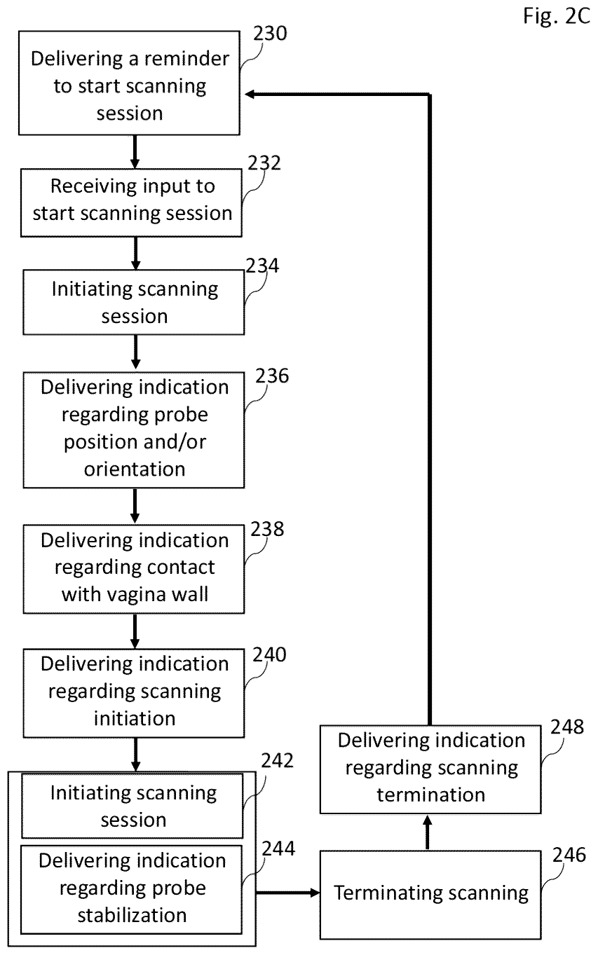

Reference is now made to FIGS. 2A and 2B, depicting scanning of a body volume by scanning sub-sectors of the body volume, according to some exemplary embodiments of the invention.

According to some exemplary embodiments, a probe, for example an ultrasound probe 202 is inserted into a body cavity, for example into a vagina 204. In some embodiments, a scanning portion 206 of the probe 202 is stabilized at a specific location within the vagina 204. Optionally, the scanning portion 206 is placed in contact with a wall of the vagina 204 at the specific location.

According to some exemplary embodiments, the scanning portion 206 of the probe is positioned and/or aimed to scan a selected body volume 208. In some embodiments, the body volume 208 comprises one or more tissues, for example organs. In some embodiments, the organs comprise at least one of, a first ovary 210, a second ovary 212, a uterus 214, a bladder 216, or any organ or tissue of the pelvic region.

According to some exemplary embodiments, a scanning beam 203 of ultrasound waves generated by the scanning portion 206 has an opening angle 207, for example a beam angle, in a range between 1 degrees and 180 degrees, for example 1 degrees and 45 degrees, 5 degrees and 70 degrees, 10 degrees and 100 degrees, or any intermediate, smaller or larger range of values. In some embodiments, the scanning beam is emitted at a specific scanning angle 205 relative to a longitudinal axis of the probe, scans a sub-sector of the body volume 208, for example sub-sector 218. In some embodiments, the scanning angle 205 is determined relative to a z-axis, for example as shown in FIG. 2B, which is optionally a longitudinal axis of the probe or a longitudinal axis of the vagina. In some embodiments, the scanning beam 203 or at least one ultrasound transducer generating the beam 203 is then moved to a different scanning angle 205. for example to scan a different sub-sector of the body volume 208, for example sub-sector 220. In some embodiments, the different sub-sector is an adjacent sub-sector to a previously scanned sub-sector. In some embodiments, two adjacent sub-sectors overlap or are spaced-apart from each other. In some embodiments, the scanning beam moves to a different scanning angle 205, optionally selected to allow adjacent scanned sub-sectors to overlap.

According to some exemplary embodiments, the scanning beam moves between different scanning angles to scan a sector of the body lumen 208 having an overall angle 210 in a range between 30 degrees and 360 degrees, for example an overall scanning angle in a range between 30 degrees and 100 degrees, between 60 degrees and 150 degrees, or any intermediate, smaller or larger range of value.

### Exemplary System-Patient Interaction

According to some exemplary embodiments, the scanning system is configured to be used by a user, for example a patient that has limited or no experience and/or knowledge in ultrasound scanning. Optionally, the scanning system is used by a subject, for example a patient, at home while introducing a scanning probe into a body cavity of the subject, for example into the vagina or rectum. In some embodiments, the canning system is configured to allow self-use by delivering indications to the subject before, during and/or after a scanning section. In some embodiments, these indications guide or instruct the subject to scan a body region, for example a pelvic region with a quality that is sufficient in order to monitor, detect and/or identify a state of tissue in the body region and/or changes thereof.

Reference is now made to FIG. 2C, depicting a system-patient interactions process in a timed relationship with a scanning section, according to some exemplary embodiments of the invention.

According to some exemplary embodiments, a scanning regime is downloaded to the scanning system, for example by an expert. In some embodiments, the scanning regime is an initial scanning regime selected by the expert, for example according to subject characteristics, physiological and/or clinical state. Alternatively, the scanning regime is selected by the user of the system out of a plurality of scanning regimes stored in a control unit of the probe or in a remote device. Optionally, the user selects and downloads a scanning regime from a remote device, for example a server or a cloud. In some embodiments, a scanning regime comprises a frequency of scanning session, duration of each scanning session, position settings of the probe within the body cavity, for example position settings determined by the expert.

According to some exemplary embodiments, the scanning system delivers a reminder to the patient to start a scanning session, at block 230. In some embodiments, the reminder comprises a human detectable indication that includes information to start a scanning session, or information regarding an initiation time and/or date of the scanning session. Optionally, the time and/or date for initiating the scanning session is based on a pre-determined program which includes two or more scanning session separated by a time interval. In some embodiments, a scanning session of the two or more scanning session is scheduled to be performed at least every 2 hours, for example at least every 6 hours, at least every 12 hours, at least every 24 hours, at least every 48 hours, at least twice a week, at least every week, at least every two weeks, at least every month, or any intermediate, smaller or larger time period.

According to some exemplary embodiments, the system receives an input signal to start a scanning session, at block 232. In some embodiments, the system receives the input signal from the user. In some embodiments, the input signal indicates compliance of the user with the planned scanning program and/or compliance of the user with the scheduled scanning session.

According to some exemplary embodiments, the system initiates a scanning session, at block 234. In some embodiments, imitating a scanning session comprises moving the system between a stand-by state to an active state. Optionally, in an active state the system receives input data from at least one sensor, for example at least one sensor of the probe. Alternatively or additionally, when moving to an active state the system performs self-calibration and/or self-check processes to ensure proper activation during the scanning session.

According to some exemplary embodiments, the system delivers an indication to a user regarding a position and/or orientation of the ultrasound probe, at block 236. In some embodiments, the indication is generated based on signals received from at least one position and/or orientation sensor of the probe. In some embodiments, the indication, for example human detectable indication, indicates whether the probe is located at a desired position within the vagina and/or if the probe orientation, for example orientation of a scanning portion of the probe relative to a target body region or a target body tissue, is a desired orientation.

According to some exemplary embodiments, the system delivers a contact indication that the probe, for example a scanning portion of the probe, is in contact with the vagina wall, at block 238. In some embodiments, the contact indication is a human detectable indication, generated by the system in response to signals received from the probe indicting sufficient contact, for example contact that is sufficient in order to acquire scan data with a desired quality, with the vagina wall.

According to some exemplary embodiments, the system delivers an indication regarding scanning initiation, at block 240. In some embodiments, the indication, indicates for the user to maintain the ultrasound probe at a desired position and/or a desired orientation with minimal or no movement of the probe during the scanning. As used here, minimal movement means movement of the probe relative to the desired position and/or desired orientation of less than 10 cm, for example movement of less than 7 cm, movements of less than 5 cm, movements of less than 3 cm, or any intermediate, smaller or larger value.

According to some exemplary embodiments, the system initiates scanning, at block 242. Optionally, during analysis the system delivers an indication, for example a probe stabilization indication, to the user regarding the stabilization of the probe during scanning. In some embodiments, the probe stabilization indication indicates if the probe movements are within a desired, for example a target, range of values which allow efficient scanning of the body region, or if the probe movements are smaller than a predetermined reference value.

According to some exemplary embodiments, the system terminates scanning, at block 246. In some embodiments, the system terminates scanning if the scanning of the body region is complete. Alternatively, the system terminates the scanning if the relative movements of the probe are larger than a target range of values or if the relative movement of the probe is larger than the predetermined reference value.

According to some exemplary embodiments, the system delivers an indication to the user when scanning is terminated, at block 246. In some embodiments, the indication comprises information regarding the reasons for scanning information. Alternatively or additionally, the indication comprises information how to proceed with the scanning session, for example how to modify at least one parameter of the scanning session, for example position and/or orientation of the probe, relaxation of body muscles during scanning, holding breathing during scanning, in order to continue with the scanning session.

### Exemplary General Probe

According to some exemplary embodiments, an ultrasound probe of the scanning system is shaped and sized to be introduced into a body cavity, for example into a vagina or a rectum of a subject, for example a female subject. In some embodiments, the ultrasound probe is configured to allow self-examination, for example self-scanning of tissue within a body cavity. by the female subject.

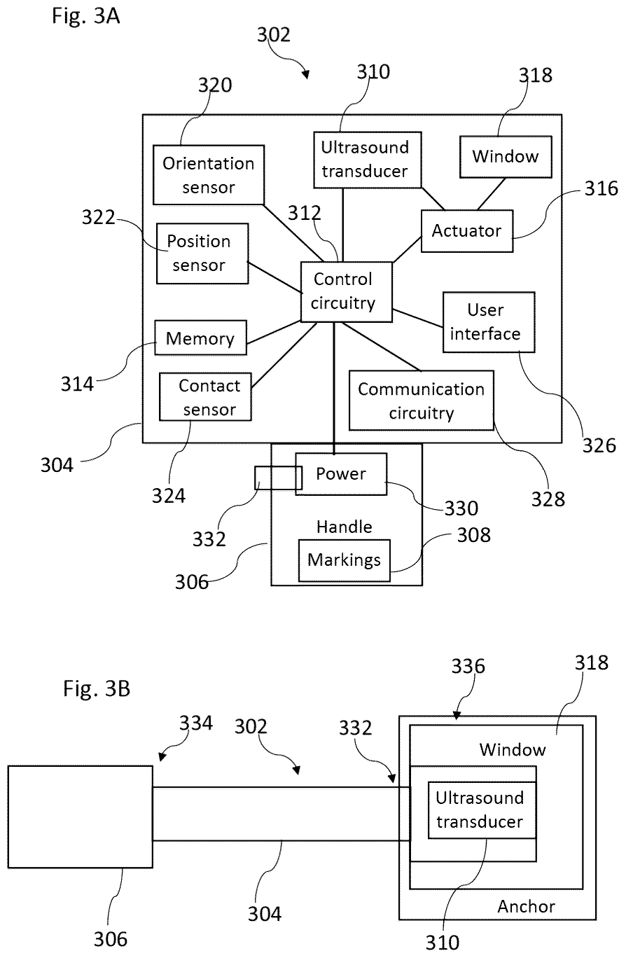

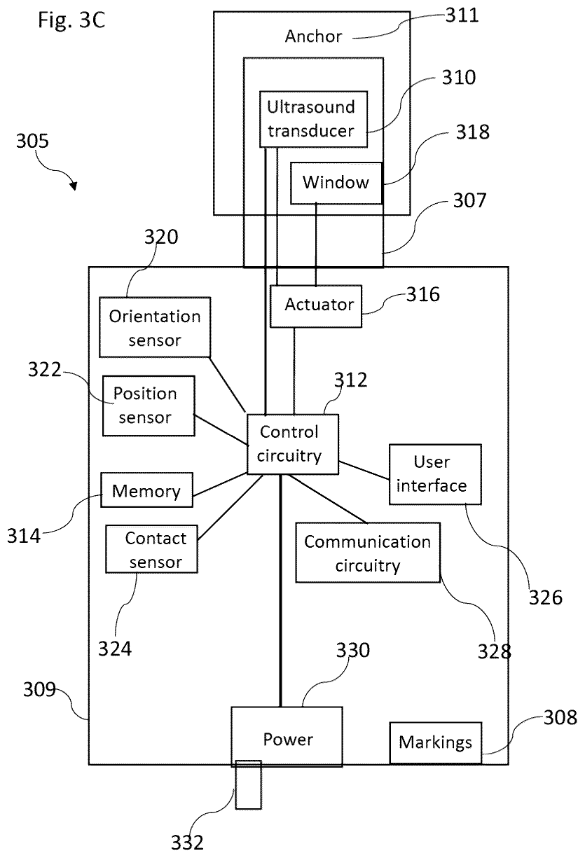

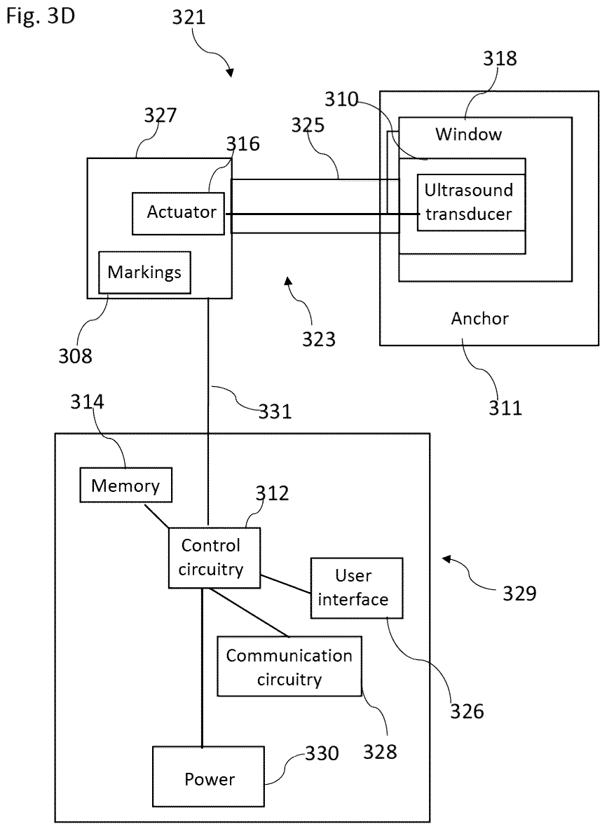

Reference is now made to FIG. 3A, depicting a general block diagram of an ultrasound probe, according to some exemplary embodiments of the invention.

According to some exemplary embodiments, an ultrasound probe, for example probe 302 comprises a body 304, for example an elongated body, shaped and sized to be introduced at least partly into a body cavity, for example an elongated body cavity, and a handle coupled to the probe body. In some embodiments, the body cavity comprises a vagina or a rectum of a subject. In some embodiments, a maximal width of the body 304 is in a range between 15 mm and 100 mm, for example in a range between 20 mm and 40 mm, 20 mm and 30 mm, 25 mm and 50 mm, 30 mm and 60 mm, 40 mm and 100 mm, or any intermediate, smaller or larger range of values. In some embodiments, an outer surface of the body 304 configured to be inserted into the body cavity is smooth, for example to prevent injury to tissue. In some embodiments, the body 304 is shaped as a tube and comprises at least one internal lumen. Optionally, the body 304 or at least part of the body is shaped as a cylinder, for example an elongated cylinder.

According to some exemplary embodiments, the body 304 has a longitudinal axis, a distal portion configured to be positioned within the body cavity during scanning, and a proximal portion, optionally configured to be positioned outside the body cavity during scanning. In some embodiments, the body 304 is straight. Alternatively, the body 304 is angled, for example between the distal portion and the proximal portion. In some embodiments, an angle between the distal portion and the proximal portion is fixed. Alternatively, the angle is adjustable, for example by an adjustable joint coupled between the distal portion and the proximal portion. In some embodiments, an angled body allows, for example, to position the distal portion of the body, optionally comprising a scanner, at a specific location within the body cavity that is not aligned with an opening of the body cavity through which the probe is inserted.

According to some exemplary embodiments, the probe 302 comprises a handle 306, optionally coupled to the body 304, for example to the proximal portion of the body 304. In some embodiments, the handle 306 comprises at least one gripping portion configured to allow holding of the probe 302 using a single hand. In some embodiments, the handle comprises one or more markings 308 for indicating an orientation of the distal portion of the body 304 located within the body cavity. Optionally, the markings indicate an orientation of a scanning portion of the probe 302. In some embodiments, the one or more markings 308 are aligned relative to the distal portion and/or the scanning portion of the probe 302. In some embodiments, the one or more markings comprise at least one of, an indentation, a slot, and a recess in the handle 306.

According to some exemplary embodiments, the probe 302 comprises at least one energy-emitting transducer, for example at least one ultrasound transducer 310. In some embodiments, the at least one ultrasound transducer 310 comprises a plurality of ultrasound transducers, optionally arranged in an array of transducers. In some embodiments, the at least one ultrasound transducer is located at the scanning portion of the probe 302, for example at the distal portion. In some embodiments, the transducer 310 is configured to emit ultrasound waves optionally with a specific opening angle, and with specific parameter values.

According to some exemplary embodiments, the probe 302 comprises a control circuitry 312, functionally coupled to the at least one ultrasound transducer 310. In some embodiments, the control circuitry 312 is functionally coupled to memory 314 in the probe 302. In some embodiments, the control circuitry 312 is configured to activate the transducer 310 to generate and emit the ultrasound waves, according to parameter values or indications thereof, stored in the memory 314.

According to some exemplary embodiments, the probe 302 comprises at least one actuator 316, for example an electric motor, functionally coupled to the ultrasound transducer 310 and the control circuitry 312. In some embodiments, the control circuitry 312 signals the actuator 316 to move the ultrasound transducer 310, for example to direct an emitting surface of the ultrasound transducer 310 towards a target sub-sector of a body volume. In some embodiments, during a scanning session, the actuator 316 changes an angle between the ultrasound transducer and the body volume, for example a scanning angle 205 of the ultrasound waves, while optionally maintaining a fixed opening angle 207 of the ultrasound beam. In some embodiments, the actuator 316 moves the transducer 310 in order to position the transducer 310 at predetermined scanning angles relative to the body volume, that were selected in order to acquire a scan image of the body volume. In some embodiments, the predetermined scanning angles were determined during a calibration process of the probe 302, and are stored in the memory 314. Optionally, the predetermined scanning angles are modified based on scan data.

According to some exemplary embodiments, the body 304 comprises at least one window 318 in the outer surface of the body 304. In some embodiments, the window 318 is positioned between the transducer 310 and the external environment outside the probe 302. In some embodiments, the at least one window is transparent to ultrasound waves, for example ultrasound waves emitted from the transducer and ultrasound waves received from tissue. In some embodiments, the window 318 limits the passage of ultrasound waves to a specific scanning angle. In some embodiments, the actuator 316 is configured to move the window 318, for example to direct the ultrasound waves at a different scanning angle relative to a previous scanning angle and/or relative to a longitudinal axis of the probe 302 or the body cavity. In some embodiments, the control circuitry 312 signals the actuator to move the window 318 based on indications stored in the memory 314, for example indications of preplanned coordinates for positioning the window 318, selected to allow scanning of a desired region of the body. In some embodiments, the actuator 318 is located in the handle 306.

According to some exemplary embodiments, the probe 302 comprises at least one orientation sensor 320, for example a gyroscope or an accelerometer, functionally coupled to the control circuitry 312. In some embodiments, the at least one orientation sensor is configured to sense changes in an orientation of the probe 302.

According to some exemplary embodiments, the probe 302 comprises at least one position sensor 322, functionally coupled to the control circuitry 312. In some embodiments, the at least one position sensor 322 is configured to sense a position, for example a relative position of the probe 302.

According to some exemplary embodiments, the probe 302 comprises at least one contact sensor 324, functionally coupled to the control circuitry 312. In some embodiments, the at least one contact sensor 324 is configured to sense contact between the probe, for example between an external surface of the probe, and a wall of the body cavity. Alternatively or additionally, the at least one contact sensor 324 is configured to sense contact between a sensing portion of the probe comprising the at least one transducer 310, and a wall of the body cavity.

According to some exemplary embodiments, the probe 302 comprises a user interface 326 configured to receive input and/or to deliver indications, to a user of the probe 302. In some embodiments, the user interface 326 is functionally coupled to the control circuitry 312. In some embodiments, the user interface 326 comprises at least one of, a speaker, a microphone, a light emitter for example a light emitting diode. In some embodiments, the user interface 326 is configured to generate one or more human detectable indications, for example audio and/or visual indications. In some embodiments, the user interface 326 is configured to allow communication between a user of the probe 302 and the probe 302. In some embodiments, the user interface 326 comprises at least one microphone for receiving voice commands from the user.

According to some exemplary embodiments, the probe 302 comprises a communication circuitry 328, functionally coupled to the control circuitry 312. In some embodiments, the communication circuitry 328 is configured to communicate, for example to transmit and/or to receive signals, with at least one different device. In some embodiments, the at least one different device comprises a close device that is located in the vicinity of the probe, for example a cellular phone, a computer, a virtual assistant device, located at a distance of up to 10 meters from the probe 302. Alternatively or additionally, the communication circuitry communicate with at least one different device comprising a a remote device, for example a cloud memory, a remote server or a remote computer, located at a distance larger than 10 meters from the probe 302.

According to some exemplary embodiments, the communication circuitry 328 is configured to send and to receive data to/from the at least one different device, for storing and/or for processing of the data in the different device. In some embodiments, the communication circuitry 328 receives signals from the different device which include modifications of the scanning process, recommendations and/or instructions to the user of the probe 302. In some embodiments, the communication circuitry transmits and/or receives wireless signals, for example Bluetooth. Wi-Fi, infra-red, or other types of electromagnetic radiation. Alternatively, the communication circuitry 328 is connected by wires to the at least one different device.

According to some exemplary embodiments, the probe 302 comprises at least one power source 330, for example a battery. In some embodiments, the at least one power source 330 is configured to provide electric energy to electrical components of the probe 302. In some embodiments, the power source 330 comprises a replaceable power source, for example, a replaceable battery. Alternatively or additionally, the power source 330 is a rechargeable power source, for example a rechargeable battery, configured to be charged from an external power source optionally via a charging connector 332.

According to some exemplary embodiments, for example as shown in FIG. 3B, the probe 302 comprises the body 304, for example an elongated body, having a distal end 332 and a proximal end 334. In some embodiments, the probe 302 comprises a scanning portion 336 which includes the at least one ultrasound transducer 310 and the window 318. In some embodiments, the window 318 surrounds at least partly the at least one ultrasound transducer 310. In some embodiments, an external surface of the scanning portion 336, for example an external surface of the window is configured to be placed in contact with a wall of the body cavity, for example to allow ultrasound contact that is sufficient for scanning of a target body region, for example a pelvic region. Optionally, the external surface of the window 318 is smooth, for example to prevent damage to the wall of the body cavity.

According to some exemplary embodiments, a proximal end 334 of the body is coupled to or comprises the handle 306.

According to some exemplary embodiments, for example as shown in FIG. 3C, a probe 305 comprises a probe body 307 shaped and sized to be at least partly inserted and positioned within the vagina, and a handle 309 configured to be positioned outside the vagina. In some embodiments, the probe 305 comprises at least one anchor, for example anchor 311, configured to stabilize the probe body 307 within the vagina. In some embodiments, the anchor 311 is an expandable anchor, configured to move, for example controllably move between a collapsed state and an expanded state. In some embodiments, the anchor 311 comprises a balloon configured to inflate and deflate, or an expandable wire frame. In some embodiments, the anchor 311 is inflated using a liquid, for example a liquid that allows passage of ultrasound waves.

According to some exemplary embodiments, the anchor 311 is positioned at least partly around the ultrasound transducer 310 and/or the window 318. In some embodiments, if the anchor 311 is a balloon it is filled with a liquid to ensure a sufficient ultrasound contact between the ultrasound transduce 310 and the tissue surrounding the probe body 307.

According to some exemplary embodiments, for example as shown in FIG. 3C, the probe body 307 comprises the at least one ultrasound transducer 310, and optionally includes a window to allow emitting of ultrasound waves generated by the at least one ultrasound transducer 310. In some embodiments, the handle 309 of the probe 305 comprises the control circuitry 312, and optionally the actuator 316. Optionally, the handle comprises at least one of, the orientation sensor 320, the position sensor 322, the memory 314, the contact sensor 324, the power source 330, the communication circuitry and the user interface 326.

According to some exemplary embodiments, for example as shown in FIG. 3D, a scanning system 321 comprises a probe 323, for example an ultrasound probe, having a body 325 and a handle 327. In some embodiments, the body 325 is optionally an elongated body which is shaped and sized to be inserted and positioned within the vagina. In some embodiments, the body 325 comprises the ultrasound transducer 310, and the window 318. Optionally the body 325 comprises the anchor 311. In some embodiments, the handle 327 coupled to the body optionally comprises the actuator 316 and/or the markings 308.

According to some exemplary embodiments, the system 321 further comprises a control unit 329, functionally coupled to the probe 323, for example by a cable or a cord, for example cord 331. In some embodiments, the control unit 329 controls the activation and/or movement of the ultrasound transducer via electrical wiring within the cord 331. In some embodiments, the control unit comprises the control circuitry 312, and the memory 314. In some embodiments, the control unit comprises at least one of, the user interface 326, a power source 330 and/or the user interface 326. Optionally, the actuator 316 is positioned in the control unit 329 and not in the handle 327, for example to reduce a weight of the probe 323.

### Exemplary System

According to some exemplary embodiments, an examination system, for example a female examination system, is used for determining a state of a tissue, located for example at the pelvic region. In some embodiments, the tissue comprises follicles, ovaries and uterus of a female subject. In some embodiments, the examination system is used to identify pathological conditions, for example endometriosis or tumors. Alternatively or additionally, the examination system is used to identify a state of the reproductive system indicating an ability of a female subject to get pregnant, for example by, determining a maturation level of a follicle and/or an oocyte within the follicle, determining a time period until ovulation and/or determining a time window of a luteal phase, using the scanning results. In some embodiments, the examination system determines a state of the tissue based on scan data received, for example, from an ultrasound probe.

According to some exemplary embodiments, the examination system defines a communication network that allows flow of information between two or more components of the system.

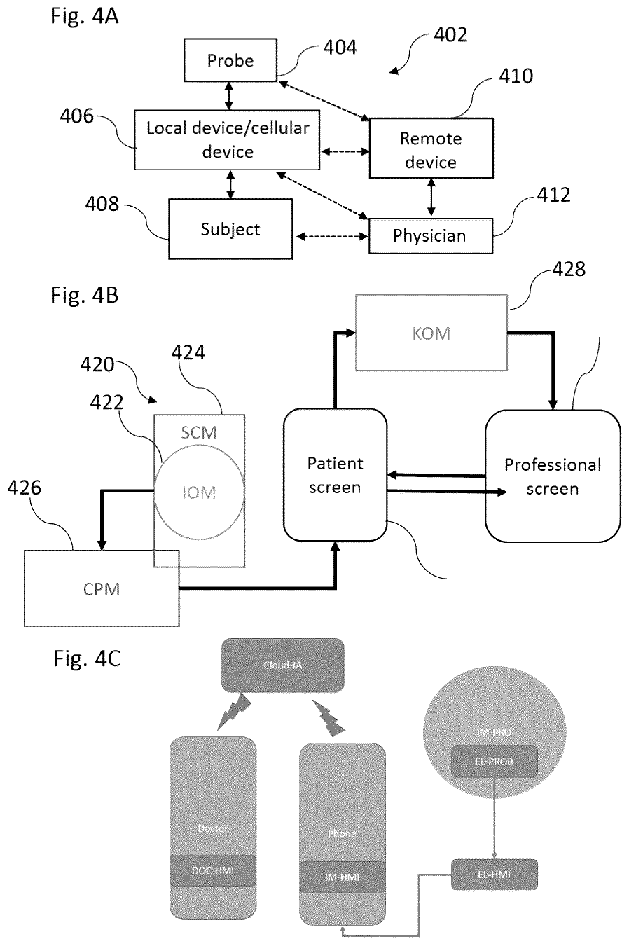

Reference is now made to FIG. 4A, depicting an examination system, according to some exemplary embodiments of the invention.

According to some exemplary embodiments, an examination system, for example system 402 comprises an ultrasound probe 404, configured to be used by a female subject, for example when receiving an indication to start a scanning session. In some embodiments, the ultrasound probe 404 comprises probe 302 shown in FIG. 3A.

According to some exemplary embodiments, the probe 404 is in communication with a local device 406, for example a cellular device, a virtual personal assistant device, a local computer, and/or a wearable device. In some embodiments, the local device 406 is located at a distance of up to 20 meters, for example up to 15 meters, up to 10 meters, up to 8 meters or any shorter or larger distance from the probe 404. In some embodiments, the probe 404 is in communication with the local device using a communication circuitry, for example communication circuitry 328 shown in FIG. 3A. In some embodiments, the probe 404 communicates with the local device 406 by wireless signals.

According to some exemplary embodiments, the local device 406 comprises a user interface that allows a subject 408, for example a female subject, to receive one or more indications, for example guidance, instructions, reminders, reports, recommendations and/or feedback, from one or more components of the system 402. Additionally, the user interface of the local device is used to receive input data by one or more components of the system 402, from the subject 408.

According to some exemplary embodiments, the system 402 comprises a remote device 410. In some embodiments, the remote device 410 comprises a remote computer, a cloud storage, and/or a remote server. In some embodiments, the remote device 410 is in communication with the local device 406. Optionally, the remote device 410 is in communication with the probe 404. In some embodiments, the remote device 410 is used for storage and/or for processing of information or data received from the local device 406 and/or from the probe 404. In some embodiments, the remote device 410 processes the data received from the probe 404 or from the local device 406 using at least one of, a software program, an algorithm, and a lookup table stored in a memory of the remote device 410.

According to some exemplary embodiments, the remote device 410 is configured to transmit one or more indications, for example guidance, instructions, reminders, reports, recommendations, feedback and/or results of processing to the local device 406. Alternatively or additionally, the remote device 410 transmits the one or more indications to the probe, for example modified coordinates and/or parameters of a scanning plan. In some embodiments, the remote device 410 transmits the one or more indications to an expert, for example a physician 412.

According to some exemplary embodiments, the physician 412 uses the information received form the remote device 410 to receive results of a scanning process performed by the subject 408. In some embodiments, the physician 412 can then provide recommendations and/or feedback based on the results, to the subject 408 directly, via the local device 406 and/or via the remote device 410.

Reference is now made to FIG. 4B depicting an examination system, according to some exemplary embodiments of the invention.

According to some exemplary embodiments, there is provided a portable device 420, to be inserted fully or partially in a human body cavity (such as but not limited to vagina and rectum), or positioned on the abdominal surface. In some embodiments, the device 420 is comprising of an image obtaining module (IOM) 422, a support and contact module (SCM) 424, a controller processing module (CPM) 426, a communication module, and a key operation module (KOM) 428. In some embodiments, the portable device 420 provided is capable of realizing man-machine interaction and between the care giver and the end-user. Optionally, the device 420 is owned by the end-user (E-U) as a personal device: validation and communication on the side of the care giver occurs through connected screens. The information could be stored in the electronic medical record (EMR) of the E-U. In some embodiments, the device is self-operated, no or minimal-manipulation needed, by non-expert hands.

According to some exemplary embodiments, the image obtaining module (IOM) 422 or “Probe” is shaped and sized for each cavity insertion and can be inserted or positioned by the end-user (E-U) themselves manually or with the help of an applicator with no guidance from any healthcare professional. In some embodiments, the module can be deployed in an origami-like fashion after the insertion or positioning; or can be kept folded for utilization. In some embodiments, the image obtaining module 422 is fitted with ultrasound transducers but can additionally or alternatively use other optical means, for example a camera or an optic sensor. In some embodiments, the module is inserted only or inserted and hold in place by the E-U but not mobilized in anyway by the E-U. Since no ultrasound training is required, in some embodiments, there is only 1 position possible to insert the module. In some embodiments, the module can be used lying down, sitting or standing according to the instructions.

According to some exemplary embodiments, the IOM 422 is mounted on a rotating system that will allow to scan the full working volume. In some embodiments, this system can be based on a 1 axis rotating mechanical system, or alternatively on a 2 degrees of freedom mechanical solution. In some embodiments, the probe will be equipped with a mechanical system that will move the probe, in a fully automated preset manner, optionally without human intervention. Optionally, the system could be equipped with adult novelty features, such as but not limited to, vibrating structures, music sound system, and pulsating lights.

According to some exemplary embodiments, the support and contact module (SCM) 424 is consisting of but not limited to, a holder in the shape of a gynecologic device (such as but not limited to a vaginal sponge, a vaginal cup, a menstrual cup, a contraceptive diaphragm, a balloon catheter, a sex-toy, a butt plug) with an expansion system, expandable with memory shape retaining material or with water or gel inflated balloons, for example, to ensure the perfect continuity between the image obtaining module 422 and the anatomical tissue of the cavity in regard to the area of interest without interspacing air. Alternatively or additionally, the SCM 424 can be sitted on a cap and a classical ultrasound gel may be added to increase the performance of the images acquisition (for example like any other echo-graphic system). In some embodiments, this module can be a consumable or included in the IOM 422 itself. Alternatively, the patient can sit on the device and the pressure thus incurred on the SCM 424 would ensure the perfect continuity via inflation into the cavity. Optionally, set movements from the patient could allow for facilitating the full volume scanning. In some embodiments, a booklet explaining the set movements could be added to the device packaging.

According to some exemplary embodiments, the controller processing module (CPM) 426 is set for acquisition and data processing received from the image obtaining module 422 in a one-step full volume pelvic scan that needs no manipulation from anyone to scan the full volume needed. In some embodiments, tt allows for a self-operated image obtaining module.

According to some exemplary embodiments, a communication module (CM) for transferring images and/or measurements to external devices, systems or databases is configured to send and receive data from one or more of: a user, a healthcare institution, a healthcare provider. In some embodiments, it is configured to send and receive data from a cellular phone. Optionally, the data processor includes a volatile memory for storing instructions and/or data, and/or a non-volatile storage, for example, a magnetic hard-disk and/or removable media, for storing instructions and/or data. Optionally, a network connection is provided as well. A display and/or a user input device such as a keyboard or mouse are optionally provided as well.

According to some exemplary embodiments, the key operation module (KOM) 428 located in a separate location independent from the E-U and the care giver. Allows for personalized image analysis. Specifically developed-in-house algorithm that automatically recognize the plane of interest, detects and measures the ultrasound images and translate them into numerical data such as but not limited to ovaries, endometrial thickness, ovarian follicles, 3D reconstruction of the follicles' volume and color code attribution according to the maturation process of the follicles also translated in a numerical data to ensure integration into the habitual workflow and into the EMR if needed.

FIG. 4C shows interactions and information flow between a phone and a doctor with a cloud, and information flow between a probe and a phone, according to some exemplary embodiments of the invention.

### Exemplary User Activities

According to some exemplary embodiments, a user, for example a female subject, uses the system to monitor a state of one or more organs of the reproductive system, for example to determine an ability of the reproductive system to initiate and/or support pregnancy in the female subject. Alternatively or additionally, the user uses the system to detect abnormalities in tissue of the pelvic region, for example to diagnose and/or to detect a pathological state of the tissue.

Reference is now made to FIG. 5A, depicting activities of a user using the system, according to some exemplary embodiments of the invention.

According to some exemplary embodiments, a user receives a scanning regime, at block 502. In some embodiments, the scanning regime is personalized according to a specific purpose of the user. In some embodiments, the scanning regime comprises a frequency of scanning sessions, and/or identity of tissue to be scanned by a probe of the system. Additionally or alternatively, the scanning regime comprises calibration information of the probe, for example one or more images or scan data acquired from a target position in a body cavity, for example the vagina or rectum, optionally when the probe has a specific orientation within the body cavity. Optionally, the calibration information is acquired in a calibration scanning process performed by an expert, for example a physician, optionally in a clinic.

According to some exemplary embodiments, the scanning regime is selected by an expert or by the user from a list of scanning regimes stored in a memory associated with the scanning system, for example a memory of the probe, for example probe 404 or probe 302, a memory of a local device, for example local device 406 or a memory of a remote device 410. Optionally, the scanning regime is downloaded to a memory of the probe or a memory of a local device in communication with the probe.

According to some exemplary embodiments, the user receives a reminder to initiate a scanning section, at block 504. In some embodiments, the reminder comprises at least one human detectable indication transmitted from at least one of a remote device, local device or the probe. In some embodiments, the reminder reminds the user to initiate a scanning session within a specific time period.

According to some exemplary embodiments, the user inserts the probe into the body cavity, for example into the vagina, at block 506. Optionally, the user inserts the probe into the vagina after applying gel on a scanning portion of the probe, for example to improve ultrasound contact with the vagina wall.

According to some exemplary embodiments, the probe, for example a distal end of the probe, is optionally positioned at the fornix, at block 508. In some embodiments, the user changes a body posture in order to direct the probe to a desired location within the vagina, for example to place the probe distal end at the fornix. In some embodiments, the distal end of the probe is introduced through the vagina and is optionally positioned at an external orifice of the cervix. In some embodiments, the distal end of the probe is placed in contact with the vagina wall at the fornix, or with the vagina wall at the cervix external orifice.

According to some exemplary embodiments, the user optionally receives indications, for example human detectable indications, regarding a position and/or orientation of the probe within the vagina, at block 510. In some embodiments, the indications include instructions how to move, advance and/or rotate the probe in order to reach a target position and/or a target orientation within the vagina. In some embodiments, the indications comprise audio and/or visual indications. In some embodiments, the indications are generated by the probe and/or by a local device in communication with the probe. In some embodiments, the indications comprise instructions to stop movement, advancement and/or rotation of the probe, for example when reaching a target position and/or a target orientation.

According to some exemplary embodiments, the user optionally receives indications regarding contact between the probe and the wall of the vagina, at block 512. In some embodiments, the indications include information regarding a quality of contact between the probe and the vagina wall. Alternatively or additionally, the indications include instructions to the user how to improve the contact. In some embodiments, the indications includes information that the probe reached a target quality level with the vagina wall.

According to some exemplary embodiments, scanning is initiated at block 514. In some embodiments, the user initiates the scanning. Alternatively the scanning is initiated automatically by the probe, for example when the probe is at a target position, at a target orientation and/or when the probe is in sufficient contact with the vagina wall.

According to some exemplary embodiments, the user maintains axial and/or angular position of the probe, during the scanning, at block 516. In some embodiments, maintaining axial position comprises maintaining an axial position of up to 3 cm from a target axial position. In some embodiments, maintaining angular position, comprises maintaining an angular position of up to 5 degrees relative to a target angular position.

According to some exemplary embodiments, the user receives indications regarding position and/or orientation of the probe, for example as described at block 510, during the scanning at block 515. Additionally or alternatively, the user receives indications regarding contact of the probe, for example as described at block 512, during the scanning at block 515. In some embodiments, the indications comprise instructions, for example audio instructions, how to change a position and/or orientation of the probe, for example to improve contact of the probe with the vagina wall.

According to some exemplary embodiments, during the scanning 515, the user receives one or more indications regarding the content of the scanned data, for example if a scanned data includes scanned data if a predetermined target tissue. Optionally, if the scanned data does not include scanned data of the predetermined target tissue, the user receives instructions, for example voice instructions how to change a position and/or orientation of the probe in order to scan the predetermined target tissue.

According to some exemplary embodiments, the user receives an indication when scanning is finished, at block 518. Optionally, the indication includes feedback regarding the scanning, for example According to some exemplary embodiments, the user removes the probe out from the vagina, at block 520.

According to some exemplary embodiments, the user receives feedback and/or instructions, at block 522. In some embodiments, the feedback includes information regarding at least one of, quality of the scanning, type of organs scanned, initial results of the scanning and/or instructions how to proceed with the scanning section. Optionally, the feedback and/or instructions are provided while the probe is still at least partly within the vagina, at block 518.

According to some exemplary embodiments, the user optionally receives instructions or recommendations to modify the scanning regime, at block 524. In some embodiments, modifying the scanning regime comprises changing a time between scanning sections and/or scanning of one or more different target tissues.

### Exemplary System Activities

Reference is now made to FIGS. 5B and 5C, depicting a process for scanning of a body volume, according to some exemplary embodiments of the invention.

According to some exemplary embodiments, calibration is optionally performed at block 528. In some embodiments, during calibration the system associates scan data with specific position and/or orientation of the probe. In some embodiments, during calibration at block 528, a probe is positioned at a specific location within a vagina and/or in a specific orientation. In some embodiments, following positioning of the probe, the probe emits ultrasound waves towards at one or more scanning angles towards a body cavity optionally including at least one target tissue, for example at least one ovary, at least one follicle, a uterus, at least one fallopian tube, and/or at least one oocyte.

According to some exemplary embodiments, the system processes the received scan data to identify the at least one target tissue in the scan data and/or other tissue, for example anatomical landmarks in the scan data. Optionally, the system uses previously acquired imaging data, for example x-ray, ultrasound, computerized tomography (CT), Magnetic Resonance Imaging (MRI), Positron emission tomography (PET), to identify tissue in the scanned data and/or during the association procedure. In some embodiments, the system associates the position and/or orientation of the probe, for example coordinates of the probe, within the vagina, with a relative position and/or orientation of the identified target tissue with respect to the probe. Alternatively or additionally, the system associates the position and/or orientation of the probe. In some embodiments, a scanning plan is generated based on the association information, for example by, determining a preferred position and/or orientation of the probe, determining the number of scans or the number of separate sub-sectors that need to be scanned in order to collect sufficient information on the target tissue. Alternatively or additionally, the scanning plan comprises a dwell time of the ultrasound beam and/or an overall duration of the scanning in each scanning session.

According to some exemplary embodiments, a plurality of scanning plans are generated, for example each for a different position and/or different orientation of the probe within the vagina of a specific subject. Optionally, one or more scanning plans are general plans that can be used by different subjects and/or when the probe is placed at different positions and or orientations within the vagina. In some embodiments, the plurality of scanning plans are stored in a memory associated with the system, for example a memory of the probe, a memory of the local device, and/or a memory of a remote device.

According to some exemplary embodiments, in the calibration 528 the scanning process is personalized for at least one of, a specific user of the probe, a specific body cavity of the user, a specific body volume that needs to be scanned, at least one target tissue, and for at least one clinical application. In some embodiments, a calibration scanning is performed by the expert. Alternatively the calibration scanning is performed by the user, for example at home. In some embodiments, during calibration the system provides indications and/or instructions how to perform the calibration and/or how to use the probe. In some embodiments, processing of the scanned data, associating the scanned data with position and/or orientation of the probe and/or generation of a scanning plan is performed by the probe and/or by a device, for example a local or a remote device that is in communication with probe.

According to some exemplary embodiments, when the probe is introduced into the vagina, the system identifies a position and/or orientation of the probe at block 520. In some embodiments, the system identifies the position and/or orientation of the probe based on the association performed during calibration. In some embodiments, the system performs at least one alignment scan when the probe is positioned within the vagina, followed by determining a relation between the results of the alignment scan and the calibration data to identify the position and/or orientation of the probe within the vagina. In some embodiments, the system determines a relation between the scanned data in the alignment scan and position and/or orientation of the probe using at least one algorithm and/or a lookup table associating scan data with position and/or orientation coordinates of the probe within the vagina.

According to some exemplary embodiments, an indication is delivered to the user regarding a position and/or orientation of the probe within the vagina, at block 532. In some embodiments, the indication includes confirmation that the probe is at a desired position and/or orientation. Alternatively, the indication includes instructions how to change a position and/or orientation of the probe in order to reach a desired, for example a target, position and/or orientation of the probe within the vagina.

According to some exemplary embodiments, the probe generates and directs at least one first ultrasound beam, at a first scanning angle, at block 534. In some embodiments, the first scanning angle is predetermined based on the previously generated scanning plan.

According to some exemplary embodiments, a 2D image of a first sub-sector is captured at block 536. In some embodiments, the 2D image is captured based on the scan data received following the generation and the direction of the first ultrasound beam at block 534.

According to some exemplary embodiments, the probe generates and directs at least one second ultrasound beam, at a second scanning angle, at block 538. In some embodiments, the second scanning angle is predetermined based on the previously generated scanning plan. Alternatively, the second scanning angle is calculated based on the scan data and/or the 2D image captured at block 536.

According to some exemplary embodiments, a 2D image of a second sub-sector is captured at block 540. In some embodiments, the 2D image is captured based on the scan data received following the generation and the direction of the second ultrasound beam at block 538.

According to some exemplary embodiments, the generation and direction of one or more additional ultrasound beam at different scanning angles followed by capturing of one or more additional 2D images, is repeated at block 542.

According to some exemplary embodiments, the system forms a 3D image of the scanned volume at block 544.

According to some exemplary embodiments, the system optionally identifies target tissue in the formed 3D image, at block 546. In some embodiments, the system selects a region of interest (ROI), within the formed 3D image, which includes the target tissue.

According to some exemplary embodiments, the system repeats capturing of additional 2D images of one or more additional selected sub-sectors in the selected ROI, for example to increase a resolution of scanning in the selected ROI.

According to some exemplary embodiments, the system optionally forms a 3D image of the selected scanned ROI, at block 550. In some embodiments, the 3D image is formed using the additional captured 2D images of the ROI.

According to some exemplary embodiments, the 3D image is transmitted to a remote device for further processing and/or storage, at block 552. Optionally, the transmitted 3D image is used to build a database that will include a plurality of scan data and/or 3D images from a single or multiple subjects.

Reference is now made to FIG. 5C, depicting a process which is similar to the process described in FIG. 5B without the generation of a 3D image by the probe or by a local device, according to some exemplary embodiments of the invention. According to some exemplary embodiments, for example as shown in FIG. 5C, the generation of a 3D image from the captured 2D images is performed in the remote device, using 2D images transmitted at block 552 to the remote device.

### Exemplary Processing of Scan Data

According to some exemplary embodiments, processing of scanned data is performed by at least one of, the probe, a local device coupled to the probe and/or a remote device. In some embodiments, for example as described in FIGS. 5B and 5C, scanned data is used by the probe and/or a local device coupled, for example wirelessly coupled, to the probe to identify that the probe is at a desired location and/or in a desired orientation within the vagina. In some embodiments, processing of scanned data to identify a state of tissue and/or to detect changes in the tissue state is performed in a remote device. Alternatively, processing of scanned data to identify a state of tissue and/or to detect changes in the tissue state is performed in the local device.

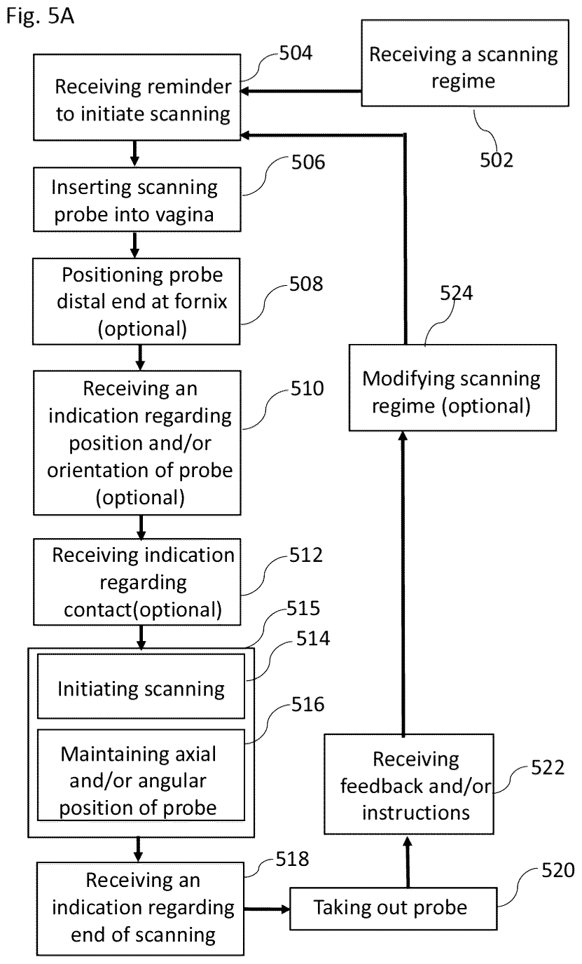

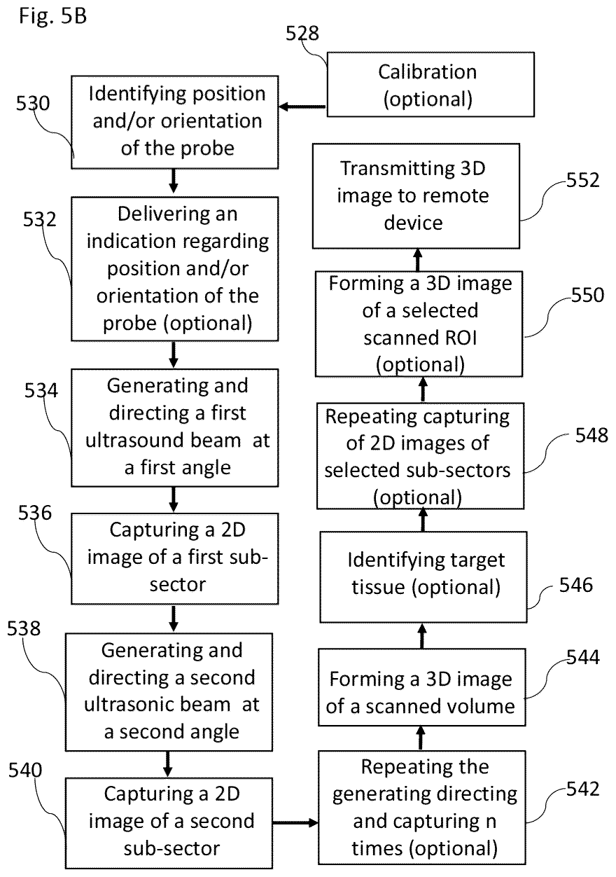

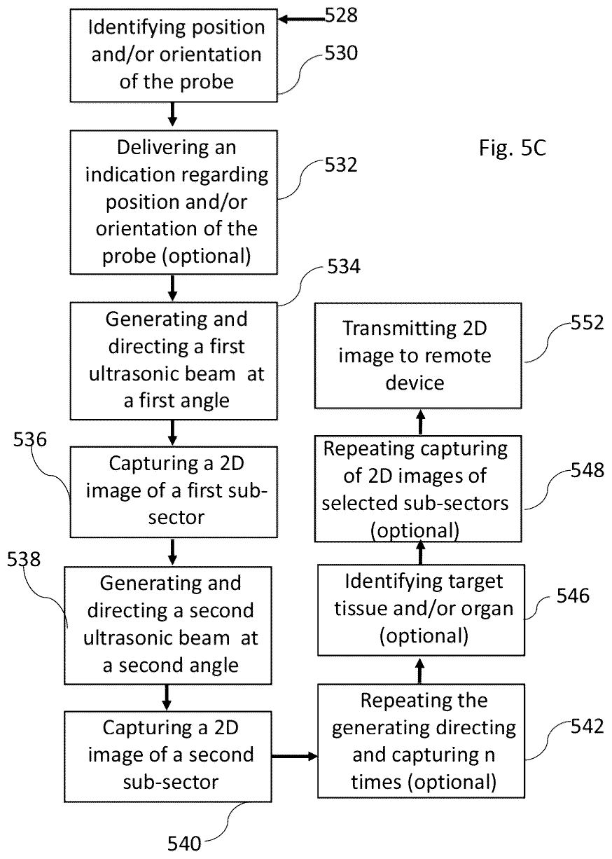

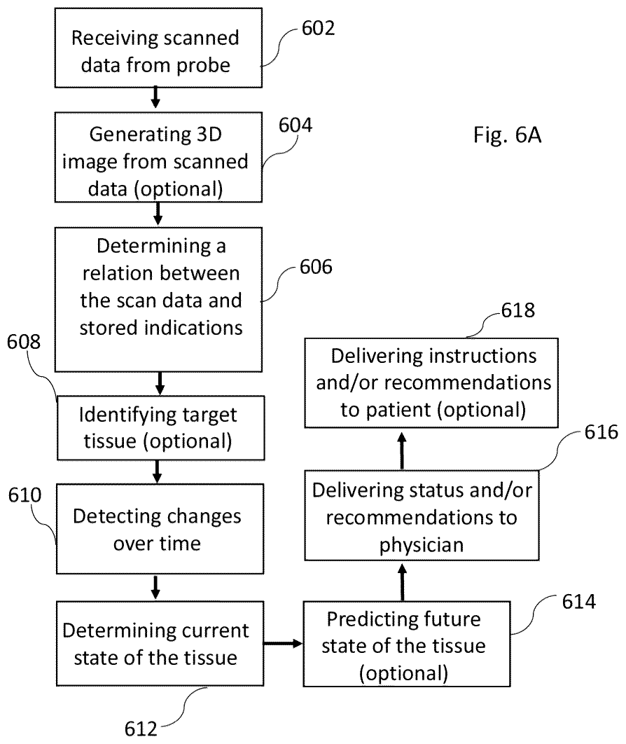

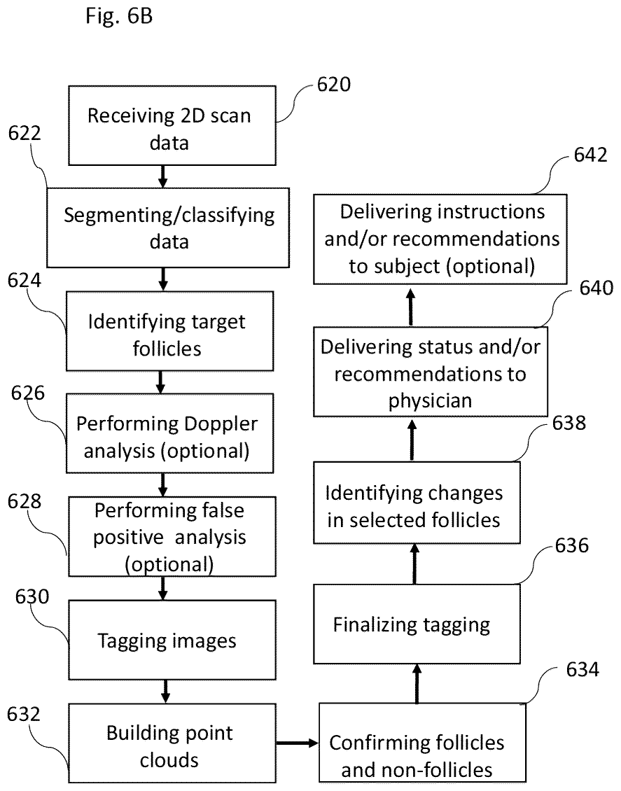

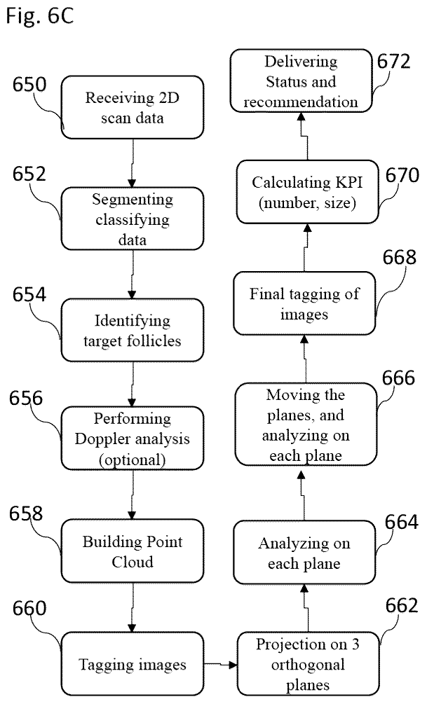

Reference is now made to FIG. 6A, depicting a general procedure for processing scan data, according to some exemplary embodiments of the invention.

According to some exemplary embodiments, scan data is received from the probe by a device, for example a local device or a remote device, at block 602. Optionally, the scan data comprises one or more 2D images.

According to some exemplary embodiments, at least one 3D image is optionally generated from the received scan data, at block 604. In some embodiments, the at least one 3D image is generated by at least one algorithm or software installed in a memory of the device.

According to some exemplary embodiments, the device determines a relation between the received scan data and one or more indications stored in a memory of the device, at block 606. In some embodiments, the indications comprise stored scan data. Alternatively or additionally, the indications comprise values of parameters that characterize one or more objects, for example tissue and/or anatomical landmarks in the scanned body volume or in the scanned body region. Optionally, the device determines a relation between the at least one generated 3D image and a 3D image or indications thereof stored in a memory of the device.

According to some exemplary embodiments, a target tissue is optionally identified at block 608. In some embodiments, the target tissue is identified based one the relation determined at block 606.

According to some exemplary embodiments, the device detects changes in the scan data, over time, at block 610. In some embodiments, the device detects changes in a target tissue over time. In some embodiments, the changes are detected based one the relation determined at block 606.

According to some exemplary embodiments, the device determines a current state of a target tissue, at block 612. In some embodiments, the current state of the target tissue is determined based on the detected changes.

According to some exemplary embodiments, the device optionally predicts a future state of the target tissue, at block 614. In some embodiments, the future state of the target tissue is predicted based on the currents state determined at block 612 and optionally using additional information, for example clinical state of the subject, social behavior of the subject, daily life of the subject, and/or drug regime of the subject.

According to some exemplary embodiments, the device delivers indications to an expert, for example a physician, based on the current and/or predicted state of the target tissue, at block 616. In some embodiments, the delivered indications comprise at least one of, current status of the target tissue, predicted status of the target tissue, recommendations how modify at least one of, a clinical state of the subject, social behavior of the subject, daily life of the subject, and/or drug regime of the subject, in order to, for example, reach a desired clinical state or a desired biological or physiological outcome.

According to some exemplary embodiments, the device optionally delivers indications to the subject at block 618. In some embodiments, the indications comprise the indications delivered to the expert at block 614. Alternatively or additionally, the indications include instructions how to change a scanning process or a scanning regime. In some embodiments, the device delivers the indications to the probe used for the scanning.

Reference is now made to FIG. 6B, depicting a detailed process for identifying changes in one or more target tissues, according to some exemplary embodiments of the invention.

According to some exemplary embodiments, a device, for example a local device or a remote device receives scan data, at block 620. In some embodiments, the scan data comprises 2D scan data or 3D scan data. In some embodiments, the device receives the scan data directly from the probe, for example a scanning probe. Alternatively, if the device is a remote device the scan data is delivered to the remote device from the probe using a local device.

According to some exemplary embodiments, the device segments and/o classifies the scan data, at block 622. In some embodiments, the device segments and/or classifies the scan data using at least one algorithm stored in a memory of the device.

According to some exemplary embodiments, the device identifies a target tissue, for example target follicles, at block 624. In some embodiments, the follicles are identified in segmented portions of the scan data or in the scan data following segmentation and/or classification.

According to some exemplary embodiments, Doppler analysis is optionally performed at block 626.

According to some exemplary embodiments, a false-positive analysis is optionally performed at block 628.

According to some exemplary embodiments, images are tagged, at block 630.

According to some exemplary embodiments, point clouds are built, at block 632.

According to some exemplary embodiments, follicles and non-follicles are confirmed in the images, at block 634.

According to some exemplary embodiments, tagging of the images is finalized at block 636.

According to some exemplary embodiments, changes in selected follicles are identified, at block 638. In some embodiments, the changes are identified in the tagged images.

According to some exemplary embodiments, indications are delivered by the device to a physician, for example to a computer of the physician. In some embodiments, the indications comprise current status and/or predicted status of the selected follicles. Alternatively or additionally, the indications comprises recommendations, for example as described at block 616.

According to some exemplary embodiments, indications, for example instructions and/or recommendations are optionally delivered to a subject using the probe, at block 642. In some embodiments, the indications include current status and/or predicted status of the selected follicles. Alternatively or additionally, the instructions include instructions described at block 642.

Reference is now made to FIG. 6C, depicting a process for analyzing 2D scan data, according to some exemplary embodiments of the invention.

According to some exemplary embodiments, 2D scan data is received at block 650.

According to some exemplary embodiments, the 2D scan data is segmented and classified at block 652.

According to some exemplary embodiments, target tissue, for example target follicles, are identified at block 654. In some embodiments, the target tissue comprises at least one of, two ovaries, two or more follicles, uterus, bladder, and/or prostate.

According to some exemplary embodiments, Doppler analysis is optionally performed at block 656.

According to some exemplary embodiments, point volume, for example point cloud is built, at block 658.

According to some exemplary embodiments, images are tagged, at block 660.

According to some exemplary embodiments, the tagged images are projected on 3 orthogonal planes, at block 662.

According to some exemplary embodiments, each plane is analyzed, at block 664.

According to some exemplary embodiments, the planes are moved and each plane is further analyzed, at block 666.

According to some exemplary embodiments, the images are finally tagged, at block 668

According to some exemplary embodiments, at least one key performance indicator (KPI), is calculated at block 670. In some embodiments, the at least one KPI comprises at least one of, number of follicles, size of follicles, shape of follicles, and position of follicles, for example relative to other follicles.

According to some exemplary embodiments, status and/or recommendations are delivered, at block 672. In some embodiments, the status and/or recommendations are delivered to an expert, for example a physician, or to the user of the probe. In some embodiments, the status and/or recommendations comprise recommendation regarding at least one of, a stimulation state of an ovary, a maturation state of a follicle, a maturation state of an egg, predicted time to egg pick up, identification of a specific at least one target follicle as a candidate for egg pick up, predicted time to ovulation.

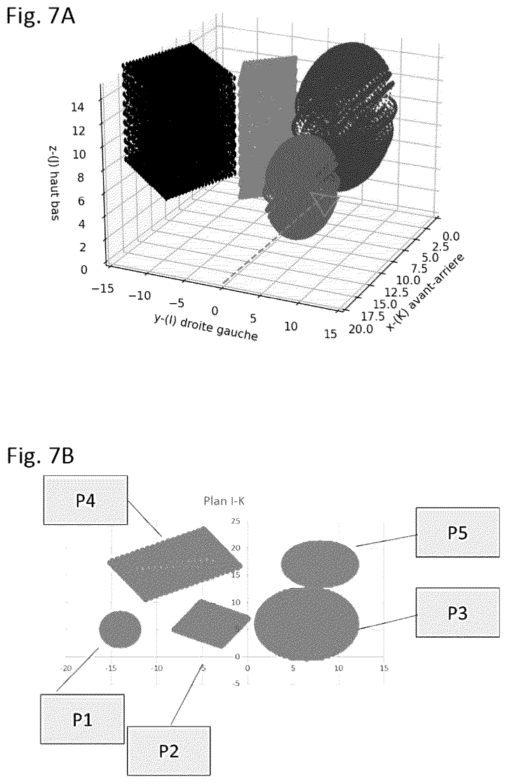

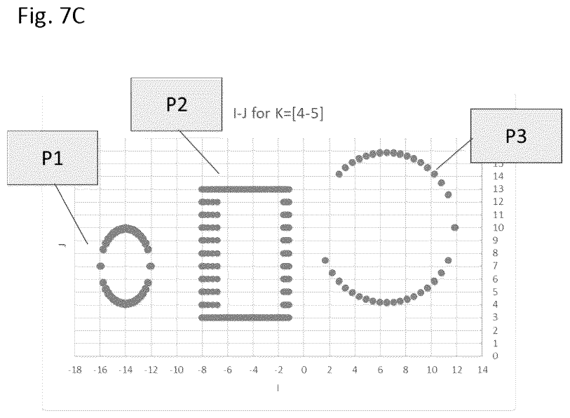

Reference is now made to FIGS. 7A-7C depicting analysis of scan data by generating a point cloud (7A), followed by analysis in two orthogonal planes (7B and 7C), according to some exemplary embodiments.

According to some exemplary embodiments, generating a point cloud, for example as described at block 658, identifies 4 objects in the point cloud, as shown for example in FIG. 7A. In some embodiments, analysis of the objects in two different orthogonal plane, for example as described at block 662-666, identifies 5 objects, as shown for example in FIGS. 7B and 7C.

### Exemplary Probe

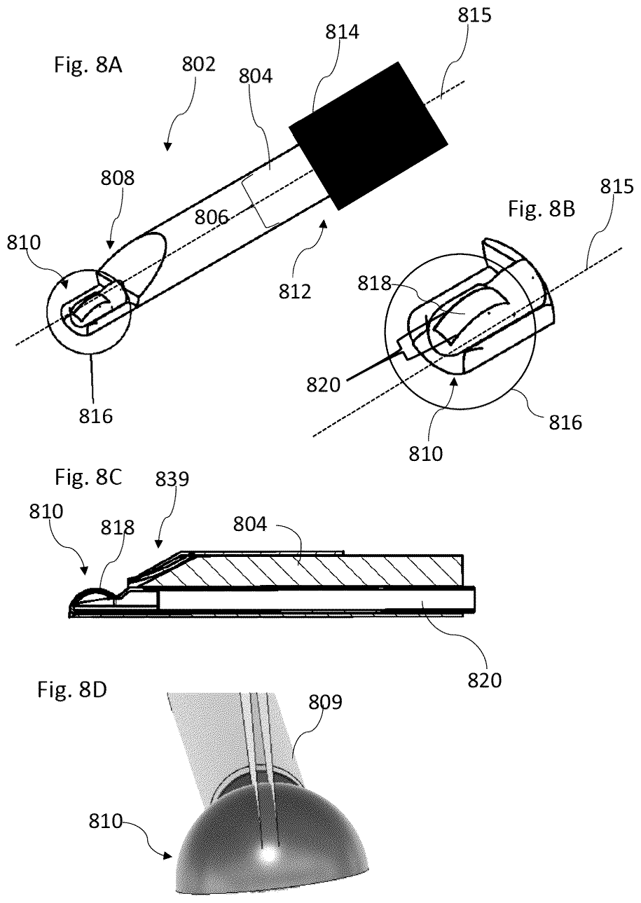

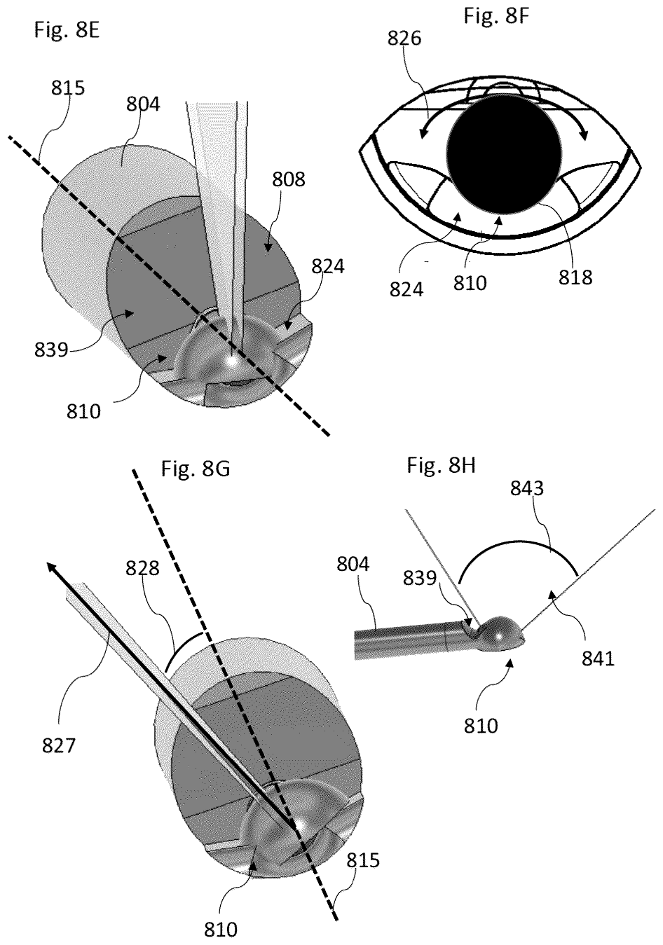

Reference is now made to FIG. 8A-8D depicting a probe, for example an ultrasound probe, according to some exemplary embodiments of the invention.

According to some exemplary embodiments, a probe, for example probe 802 comprises an elongated body 804 shaped and sized to penetrate at least partly into an elongated body cavity, for example into a vagina of a female subject. Alternatively or additionally, the elongated body is shaped and sized to penetrate at least partly into a rectum of a female or male subjects. In some embodiments, a maximal width of the body 804 is in a range between 10-100 mm, for example between 10-50 mm, between 20-80 mm, between 50-100 mm or any intermediate, smaller or larger range of values. In some embodiments, an external surface of the body 804 is smooth, for example to prevent damage to tissue of the body cavity.

According to some exemplary embodiments, the elongated body 804 has a long axis 815, a distal portion 808 comprising a scanner 810, and a proximal portion 812 comprising a handle, which is shaped and sized to be positioned outside the elongated body cavity, outside the body. In some embodiments, for example as shown in FIG. 8C, the elongated body 804 comprises at least one inner lumen 820 in which electrical wiring and/or mechanical transmission functionally couple the scanner 810 and one or more components in the handle 814, for example an actuator, a control circuitry or power source. In some embodiments, the scanner 810 is a scanning portion of the probe and is located within the probe body, at the distal portion of the probe body. Alternatively, for example as shown in FIG. 8D, the scanner 810 is located distally to the probe body 809 and optionally extends out from the probe body, for example from the distal portion of the probe body. In some embodiments, the scanner comprises at least one ultrasound transducer, and optionally a plurality of ultrasound transducers. Optionally, the plurality of ultrasound transducers are arranged as an array in said scanner, for example scanner portion of the probe. Optionally, the scanner is movable, for example relative to the probe body. Alternatively, the at leats one ultrasound transducer moves relative to the probe body.

Reference is now made to FIG. 8B, depicting a scanner of a probe, according to some exemplary embodiments of the invention. In some embodiments, a maximal width of the scanner is smaller than a maximal width 806 of the body 804. In some embodiments, the scanner 810 comprises at least one ultrasound transducer, for example a plurality of ultrasound transducers optionally arranged in an array. In some embodiments, the scanner 810 comprises at least one lens. In some embodiments, the scanner 810 comprises at least one window, for example a sideways facing window 818 transparent to ultrasound waves, which is configured to allow passage of ultrasound beams having an opening angle between 0 and 160 degrees relative to a long axis 815 of the body 804, for example an opening angle between 0 and 120 degrees, an opening angle between 0 and 90 degrees or any intermediate, smaller or larger range of values. In some embodiments, the window 818 is positioned or is part of an external cover of the scanner 810, optionally surrounding the at least one ultrasound transducer. In some embodiments, the window 818 is curved, and has a narrow width 820 which is in a range between 0.1 mm and 10 mm, for example in a range between 0.1 mm and 1 mm, in a range between 0.5 mm and 5 mm, in a range between 1 mm and 10 mm or any intermediate, smaller or larger range of values.

According to some exemplary embodiments, the size and shape of the window define an opening angle and width of the ultrasound beam, for example a narrow width ultrasound beam having an opening angle in a range between 30 degrees and 160 degrees, for example in a range between 45 degrees and 100 degrees, in a range between 60 degrees and 110 degrees, or any intermediate, smaller or larger range of values, for example as shown in FIG. 8D. Optionally, the size and shape of the window is controllably modified in order to define different dimensions of the ultrasound beam.

Reference is now made to FIGS. 8E-8H, depicting a probe with a scanner, for example a scanner portion, according to some exemplary embodiments of the invention.

According to some exemplary embodiments, a scanner 810 comprises at least one ultrasound transducer. Optionally, the scanner comprises an optics assembly. In some embodiments, the scanner 810 is functionally coupled to an actuator, for example a motor optionally located in the body 804 or in the handle 814 coupled to the body. Alternatively, the actuator is located within a control unit functionally coupled to the scanner and/or to the at least one ultrasound transducer within the scanner. In some embodiments, the scanner 810 is movable, for example rotatable. In some embodiments, the motor is configured to rotate scanner 810 within a groove or a socket 824 in the distal portion 808 of the body 804, for example as shown in FIGS. 8E and 8F.

According to some exemplary embodiments, the motor is configured to rotate, for example roll the scanner 810, or the at least one ultrasound ransducer, on a plane that is placed in an angle relative to the long axis 815 of the probe. Optionally, the scanner 810 is rolled sideways relative to the long axis 815. In some embodiments, the scanner 810 is rotated along an arc-shaped path subtending an angle between 45 degrees and 180 degrees, for example between 60 degrees and 120 degrees or any intermediate, smaller or larger range of angles.

According to some exemplary embodiments, for example as shown in FIGS. 8G and 8H, rotating the scanner 810 rotates the window and an ultrasound beam 827 passing through the window 818 in an angle 828 that is in a range between −90 degrees and +90 degrees relative to the long axis 815, for example in a range between −60 degrees and +60 degrees, in a range between −45 degrees and +45 degrees relative to the long axis 815, or any intermediate, smaller or larger range of values.

According to some exemplary embodiments, for example as shown in FIGS. 8C, 8E and 8H, the scanner 810 is positioned within a groove or a socket within the distal portion of the probe body. Optionally, the scanner is positioned distally to a slope 839 or a tapered region of the probe body, for example to prevent blockage of a scanning beam 841 of ultrasound waves, which has an opening angle 843 between 30 degrees and 180 degrees, for example between 30 degrees and 90 degrees, between 50 degrees and 90 degrees or any intermediate, smaller or larger angle.

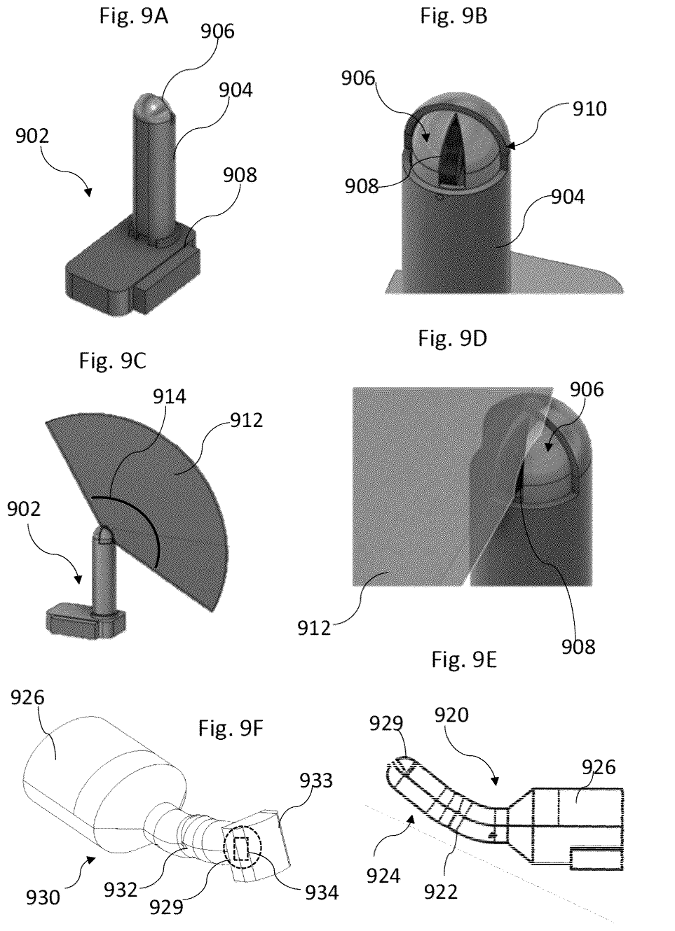

Reference is now made to FIGS. 9A-9D, depicting a probe having a movable scanner, according to some different exemplary embodiments of the invention.

According to some exemplary embodiments, a probe 902 comprises a body 904 having a proximal portion comprising a scanner 906 and a distal portion comprising or coupled to a handle 908. In some embodiments, for example as shown in FIG. 9B, the scanner 906 comprises a window 908 positioned in the scanner cover. In some embodiments, the scanner 906 is movable within a socket 910. In some embodiments, an ultrasound beam 912 is emitted from the window 908, for example as shown in FIGS. 9C and 9D. In some embodiments, the ultrasound bam has an opening angle 914 in a range between 90 degrees and 180 degrees, for example in a range between 120 degrees and 160 degrees, or any intermediate, smaller or larger range of values.

According to some exemplary embodiments, the body of the probe, for example body 804 and 904 is straight. Alternatively, for example as shown in FIG. 9E, a probe 920 comprises a curved body 922, positioning a distal portion 924 of the body 922 comprising the scanner 929 at an angle relative to the handle 926. In some embodiments, an angle between the scanner and the handle is in a range between 0.5 degrees and 90 degrees, for example in a range between 10 degrees and 50 degrees, in a range between 20 degrees and 45 degrees. In some embodiments, having a curved, for example angled body, allows for example to better insert and position the scanner at a desired position within the body cavity, by having a body that is shaped to fit into a curved body cavity.

Reference is now made to FIG. 9F, depicting a probe with an anchor, for example an expandable anchor, according to some exemplary embodiments of the invention. According to some exemplary embodiments, a probe 930 comprises a body 932 and a handle 926 coupled to a proximal portion of the body, and a scanning portion, for example a scanner 929 at a distal portion of the body 932. In some embodiments, the probe 930 comprises at least one anchor 933, coupled to the probe, for example to the probe body 932 or to the scanner portion 929 of the probe 930. In some embodiments, the anchor 933 is configured to move between an expanded state and a collapsed state. In some embodiments, during the insertion of the probe into the body cavity, the anchor 933 is in a collapsed state, for example not to interfere with the insertion process. In some embodiments, when reaching a target position within the body cavity, the anchor 933 expands to an expanded state, for example to stabilize the probe body and/or the scanner within the body cavity.

According to some exemplary embodiments, the anchor 933, surrounds at least partly the scanner 929 and/or the at least one ultrasound transducer 934 within the scanner. In some embodiments, the anchor 933 is at least partly or completely transparent to ultrasound waves. In some embodiments, during the emitting of the scanning beam, the anchor 933 is in an expanded state. In some embodiments, the anchor 933 comprises an inflatable balloon, which moves into an expanded, for example inflated state when filled with fluid or gel that allows passage of ultrasound waves.

### Exemplary Positioning and Scanning

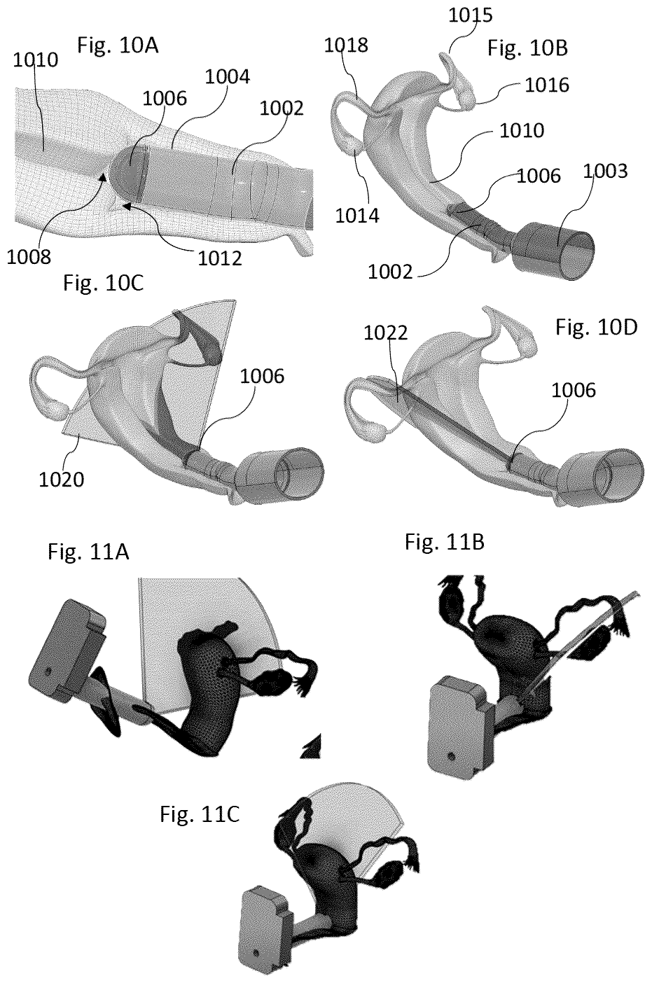

Reference is now made to FIGS. 10A-10B, depicting positioning of a probe scanner within the vagina, according to some exemplary embodiments of the invention.

According to some exemplary embodiments, a probe body 1002 is introduced into the vagina of a female subject, to position a proximal portion of the probe comprising a scanner 1006, close to an orifice 1008 of the uterus 1010. Optionally, the scanner 1006 is at least partly placed in contact with the orifice 1008. Alternatively, the scanner 1006 is placed in contact with the vagina wall at a fornix region 1012. Alternatively, the scanner 1006 is positioned within the vagina at a location in a distance smaller than 10 mm, for example smaller than 7 mm, smaller than 5 mm, smaller than 3 mm, from the fornix 1012 and/or the uterus orifice 1008.

According to some exemplary embodiments, for example as shown in FIG. 10B when the scanner is positioned within the vagina, the handle 1003 of the probe remains outside the body. In some embodiments, the body 1002 of the probe is too wide to penetrate through the orifice 1008 into the uterus 1010. In some embodiments, the vagina wall stretches and tightens at least partly around the body 1002 to the body external surface, when the probe body 1002 is inserted and positioned inside the vagina 1004.

According to some exemplary embodiments, the scanner is positioned within the vagina to allow scanning of a target tissue, for example a target tissue within the pelvic region. In some embodiments, the target tissue comprises the uterus 1010, a first ovary 1014, a second ovary 1016, a first fallopian tube 1015 and a second fallopian tube 1018.

According to some exemplary embodiments, for example as shown in FIG. 10C, when scanning is initiated, the scanner 1006 emits an ultrasound beam 1020 at a first direction, for example at a first scanning angle. In some embodiments, for example as shown in FIG. 10D, the scanner 1006 then moves to at least one different angular position, and emits at least one additional scanning beam 1022 at a different scanning angle. In some embodiments, the scanner moves to a different angular position before emitting a scanning beam. Alternatively, the scanner emits a scanning beam while moving between different angular positions.

According to some exemplary embodiments, if a scanner comprises a phased array of ultrasound transducers, then an ultrasound beam is emitted at different scanning angles without a need to use an actuator and/or without moving the scanner.

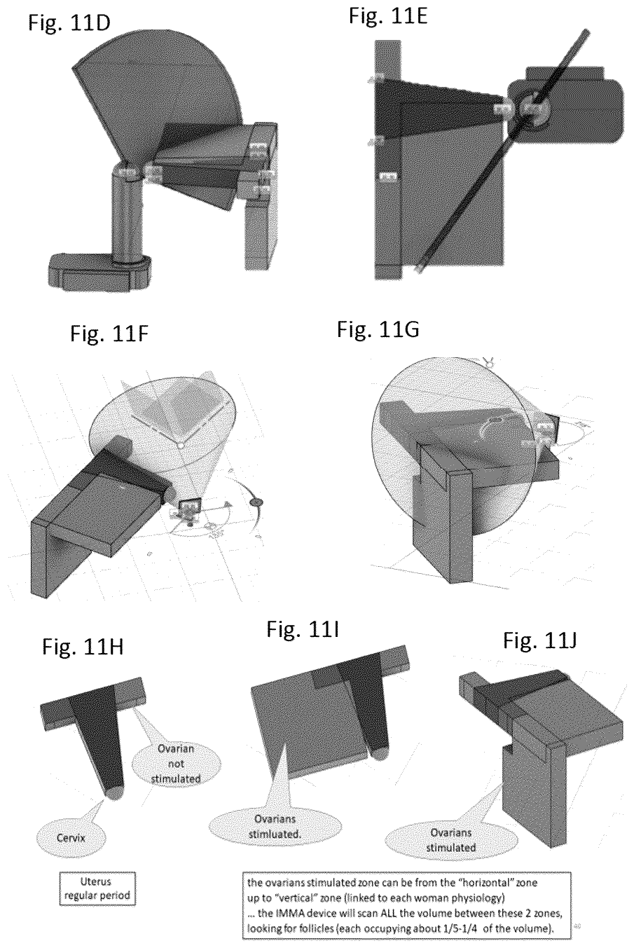

FIGS. 11A-11G are additional schematic illustrations showing a scanner of a probe emitting ultrasound beams at different scanning angles, and/or at different directions towards a tissue volume of a pelvic region, according to some exemplary embodiments of the invention.

FIGS. 11H-11J are schematic illustrations showing scanning of the ovaries, according to some exemplary embodiments of the invention.

According to some exemplary embodiments, for example as shown in FIGS. 11H and 11I. the scan data allows to distinguish between a stimulated ovary to a non-stimulated ovary. In some embodiments, for example as shown in FIG. 11J, the ultrasound beams delivered by the scanner are directed to scan a volume between two planes that are perpendicular to each other.

### Exemplary Clinical Applications

According to some exemplary embodiments, the system described herein is used as an examination system, for example for detection of a pathological state or a physiological state, for detection of a disease, and/or for monitoring a state of one or more tissues in the body over time. In some embodiments, the system is used during an in-vitro fertilization procedure to detect and/or monitor changes in one or more tissues of the reproductive system, for example an ovary, a uterus, an oocyte, a follicle, a fallopian tube.

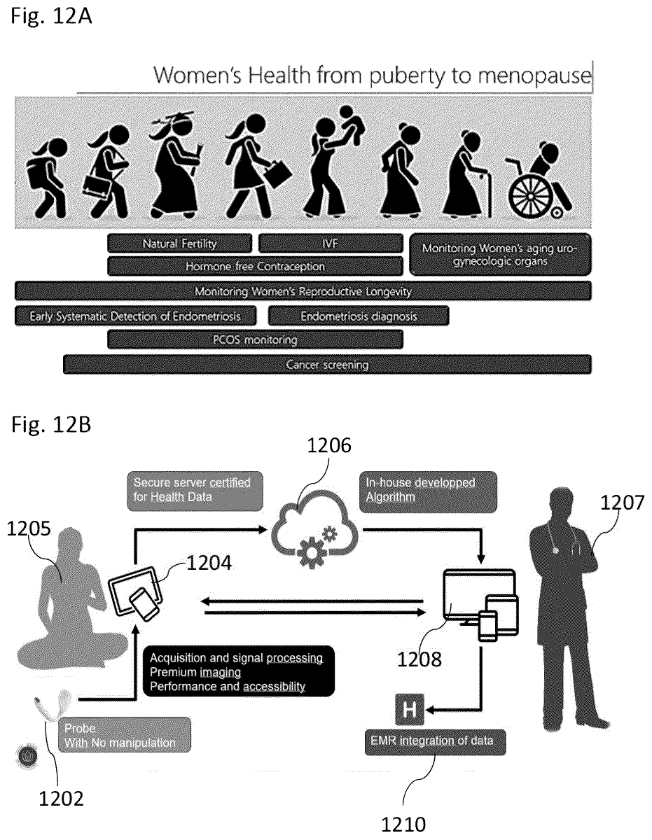

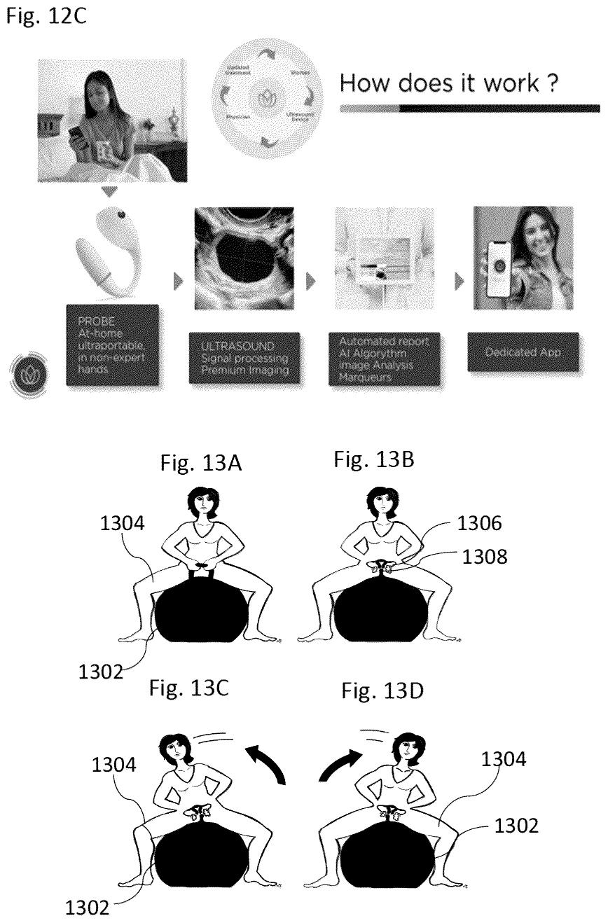

Reference is now made to FIG. 12A depicting situations in which the system is used, according to some exemplary embodiments of the invention.

According to some exemplary embodiments, the system is used to monitor a woman health from puberty to menopause.

According to some exemplary embodiments, the system is used in order to monitor natural fertility. In some embodiments, the system is used by a fertile woman to monitor a state of one or more tissues of the reproductive system. In some embodiments, the system is used to provide indications and/or predictions regarding at least one of, number of eggs in an ovary, maturation of an egg within a follicle, release of an egg from a follicle, fertilization, implantation of a fertilized egg in the uterus, and development of the fertilized egg. In some embodiments, the system provides recommendations or instructions how to modify behavior, drug regime, physical activity, at least one therapeutic process associated with the female subject based on scanning and/or the provided indications and/or predictions.

According to some exemplary embodiments, the system is used during an IVF procedure, for example to monitor ovarian stimulation. In some embodiments, the system provides recommendations or instructions how to modify a drug regime given to the female subject based on a state of the ovary, follicles and/or eggs.

According to some exemplary embodiments, the system is used for hormone-free birth control (contraception). In some embodiments, the system is used to provide indications and/or predictions regarding at least one of, maturation of an egg within a follicle and/or release of an egg from the ovary. In some embodiments, based on the system, a female subject can define a safe time-period in which fertilization is not likely to occur.

According to some exemplary embodiments, the system is used to monitor women's reproductive longevity. In some embodiments, the system provides indications regarding a current state of a woman reproductive system and predictions regarding a future state of the reproductive system. In some embodiments, the system provides predictions regarding potential to have one or more of, miscarriage, pregnancy complications, fertilization complications, a birth defect, a baby with a genetic disorder, for example down syndrome, based on a current or predicted state of the reproductive system.

According to some exemplary embodiments, the system is used to early detect or to diagnose endometriosis.

According to some exemplary embodiments, the system is used to detect, diagnose and monitor polycystic ovarian syndrome (PCOS).

According to some exemplary embodiments, the system is used for cancer detection or screening.

According to some exemplary embodiments, the system is used for monitoring aging of a woman, for example aging of gynecologic organs.

Reference is now made to FIG. 12B, depicting system components and interactions between the system components, according to some exemplary embodiments of the invention.

According to some exemplary embodiments, a system comprises a probe 1202, for example a portable probe, a software program installed in a memory of a local device 1204 of a user 1205, and a remote device 1206, for example a server, a cloud memory. In some embodiments, the user 1205 activates the probe in one or more scanning sessions of a scanning program stored in the local device 1204, for example in the software program. In some embodiments, scan data from the probe 1202 is transmitted to the local device 1204, and is optionally processed by the local device 1204. In some embodiments, the scan data or processed data is transmitted from the local device 1204 to the remote device 1206, for further processing. In some embodiments, the remote device 1206 processes the data received from the local device 1204 using at least one algorithm, formula or a lookup table stored in the memory of the remote device 1206. Optionally, all the processing of the scan data acquired by the probe 1202 is performed in the remote device.

According to some exemplary embodiments, the remote device 1206 transmits information with indications regarding the processed scan data to a local device 1208 of an expert 1207, for example a physician. In some embodiments, the physician transmits reports, updates, recommendations and/or instructions to the patient 1205 local device 1204 or directly to the patient. Alternatively or additionally, the remote device 1206 transmits instructions and/or recommendations to the patient 1205 via the local device 1204, optionally without a need of an expert.

According to some exemplary embodiments, the remote device 1206 transmits to the local device 1204 and/or to the probe 1202, optionally via the local device 1204, software updates, recommendations for alterative and/or complementary therapies, optionally personalized to a state of the user 1205 based on the processing of the scan data. In some embodiments, the remote device 1206 is used to generate a database which includes data, for example scan data and clinical data, collected from a plurality of users. In some embodiments. indications and recommendations delivered to the user 1205, for example via the local device 1204, are based on an analysis of the data collected from the plurality of users.

According to some exemplary embodiments, an expert 1207 delivers information, for example scan data, processed scan data, medical records of the user to Electronic Medical Records (EMR) systems, for example to allow EMR integration.

Reference is now made to FIG. 12C, depicting a process for using a software program, for example an application software (app), to receive information based on scan data acquired by a portable probe, according to some exemplary embodiments of the invention.

According to some exemplary embodiments, a user, for example a female subject, uses a portable probe for self-examination of the pelvic region. In some embodiments, the probe is a transvaginal portable ultrasound probe. In some embodiments, scan data acquired by the probe is processed using one or more algorithms, for example artificial intelligence algorithms, for image analysis and/or for generating recommendations and predictions based on the scan data. In some embodiments, the user receives the recommendations, predictions and indications regarding a state of scanned tissue via a dedicated software application. In some embodiments, the software application is used for communication between a physician and the user, and/or for communication between a remote device and the user.

### Exemplary Scanning Using Body Movements

According to some exemplary embodiments, the probe is coupled to an external surface while positioned within the body cavity. In some embodiments, the external surface comprises a probe support, for example an applicator that is attached to the probe outside the body and allows mechanical stabilization to the probe position within the body cavity. In some embodiments, scanning of a body region, for example the pelvic region, is performed while the probe is coupled to the external surface, using movements of the subject body. In some embodiments, scanning using body movements allows, for example, to have an ultrasound probe without a moving mechanism for the movement of a scanner or at least one ultrasound transducer, by acquiring scan data at different angles based on body movements

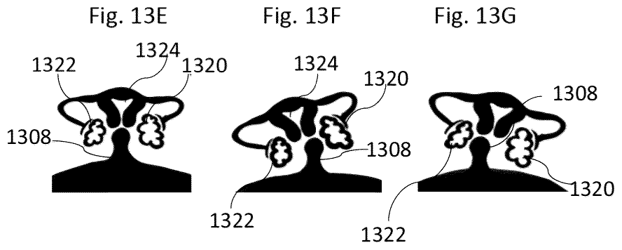

Reference is now made to FIGS. 13A-13D, depicting scanning of a body region using body movements, according to some exemplary embodiments of the invention.

According to some exemplary embodiments, a probe is coupled to an external surface 1302, for example a chair or a ball, on which a subject 1304 sits while the probe body is positioned at least partly within the vagina of the subject. In some embodiments, the external surface is in contact with the floor or a wall, for example to stabilize the external surface in at least one axis. In some embodiments, for example as shown in FIG. 13B, when the subject sits on the ball while the probe is within the vagina, the probe body, for example a scanner of the probe is located at a close distance to reproductive system tissue 1306 of the subject. In some embodiments, sitting on the probe 1308 prevents axial and/or lateral movement of the probe 1308 within the vagina.

According to some exemplary embodiments, for example as shown in FIGS. 13C and 13D, movement of the subject body, for example lateral movement and/or rotational movement of the subject body, changes a position and/or orientation of the reproductive system tissue relative to the probe body.

According to some exemplary embodiments, for example as shown in FIGS. 3E to 13G, movement of the body prior to or during scanning, for example moving of the upper part of the body changes a position and/or orientation of at least one ovary, for example ovaries, and/or uterus 1324 relative to the probe. In some embodiments, movements of the body, changes the position and/or orientation of pelvic region tissue or tissue surrounding the body cavity, relative to the probe that is stabilized within the body cavity.

It is expected that during the life of a patent maturing from this application many relevant ultrasound probes will be developed; the scope of the term ultrasound probe is intended to include all such new technologies a priori.

As used herein with reference to quantity or value, the term “about” means “within ±10% of”.

The terms “comprises”, “comprising”, “includes”, “including”, “has”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular forms “a”, “an” and “the” include plural references unless the context clearly dictates otherwise. For example, the term “a compound” or “at least one compound” may include a plurality of compounds, including mixtures thereof.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

In addition, any priority document(s) of this application is/are hereby incorporated herein by reference in its/their entirety.

1. A method for scanning a pelvic region, comprising:
stabilizing a probe within a body cavity comprising a vagina of a subject;
scanning a volume of a body region by a scanner of said probe without moving said probe relative to said body cavity; wherein an actuator moves the probe to scan the volume with a scanning angle in a range of at least 50 degrees.

2. A method according to claim 1, wherein said scanning comprising scanning said body volume by emitting an ultrasound beam towards said body region volume from said scanner, wherein said scanner comprises at least one ultrasound transducer.

3. A method according to claim 1, comprising:
processing scan data acquired during said scanning; and
identifying in said processed scan data of said body volume, reproductive tissue and/or changes thereof.

4. A method according to claim 3, wherein said processing comprises determining a relation between said scan data to previously acquired scan data or indications thereof, and wherein said identifying comprises identifying said reproductive tissue and/or changes thereof based on said determined relation.

5. A method according to claim 4, wherein said determining comprises comparing said scan data to previously acquired scan data or indications thereof, and wherein said identifying comprises identifying said reproductive tissue and/or changes thereof based on said comparison results.

6. A method according to claim 3, wherein said reproductive tissue comprises at least one ovary of said subject; and/or wherein said reproductive tissue comprises at least one follicle and wherein said identifying comprises identifying changes in at least one parameter of said at least one follicle and/or at least one parameter of said at least one ovary.

7. (canceled)

8. A method according to claim 6, wherein said identified changes in said at least one parameter of said at least one follicle and/or at least one parameter of said ovary indicating a level of stimulation or maturation of said at least one follicle.

9. A method according to claim 8, wherein said at least one parameter of said at least one follicle comprises at least one of, size, shape, external surface texture and/or position of said at least one follicle; and/or wherein said at least one parameter of said at least one ovary comprises a number of follicles in said at least one ovary, position of said at least one ovary and/or size of said at least one ovary.

10. (canceled)

11. A method according to claim 1, wherein said stabilizing comprises stabilizing said scanner of said probe within said body while said subject sits on said probe, and wherein said method comprises moving an upper part of a body of said subject during said sitting, prior to and/or during said scanning.

12. A method according to claim 1, comprising:
fixing said probe to an external surface located outside said body cavity prior to said stabilizing; and/or moving said scanner relative to said probe prior to, during and/or following said scanning.

13. (canceled)

14. A method according to claim 1, wherein said stabilizing comprising stabilizing a scanner of said probe at a distance smaller than 5 cm from an external cervix orifice or from a vaginal fornix and/or wherein said stabilizing comprises reversibly expanding an anchor coupled to said probe within said body cavity.

15. (canceled)

16. A system for scanning of a body volume, comprising:
an intracavity ultrasound probe having an elongated body shaped and sized to be introduced at least partly into a body cavity of a subject, wherein said probe comprises:
a scanner comprising at least one ultrasound transducer configured to generate an ultrasound beam and to acquire scan data;
an actuator;
a control device in communication with said intracavity ultrasound probe, comprising a memory and a control circuitry, wherein said control circuitry is configured to control the actuator to move the intracavity ultrasound probe and determine if said scan data comprises information on a target tissue, by determining a relation between said scan data received from said probe and one or more indications stored in said memory.

17. A system according to claim 16, wherein said one or more indications comprise a model of said target tissue and/or previously acquired scan data.

18. A system according to claim 16, wherein said control unit comprises a user interface configured to generate an alert signal if said scan data does not comprises information on said target tissue; and/or wherein said probe comprises an anchor coupled to said probe body and configured to move between a collapsed state and an expanded state, and to anchor said elongated body within said cavity in said expanded state.

19. (canceled)

20. A system according to claim 18, wherein said anchor surrounds at least partly said at least one ultrasound transducer; and/or wherein said anchor surrounds at least partly said elongated body; and/or wherein said anchor comprises a balloon, and wherein in an expanded state said balloon is filled with a fluid that allows passage of said ultrasound beam towards a wall of said body cavity in contact with said anchor.

21. (canceled)

22. (canceled)

23. A system according to claim 20, wherein said control circuitry signals said at least one ultrasound transducer to generate and emit said ultrasound beam when said anchor is in an expanded state.

24. (canceled)

25. A system according to claim 16, wherein said probe comprises a handle coupled to said elongated body, and the actuator is located in said handle, wherein said control circuitry signals said actuator to move said scanner and/or said at least one ultrasound transducer prior to and/or during the generation of said ultrasound beam; and/or wherein said control circuitry signals said actuator to move said scanner and/of said at least one ultrasound transducer prior to and/or during the generation of said ultrasound beam.

26. (canceled)

27. A system according to claim 25, wherein said actuator comprises an electric motor; and/or wherein said at least one actuator comprises a preloaded actuator.

28. (canceled)

29. A system for scanning of a pelvic region, comprising:
a transvaginal ultrasound probe having an elongated body shaped and sized to be introduced at least partly into a vagina of a female subject, wherein said probe comprises:
a scanner comprising at least one ultrasound transducer configured to generate an ultrasound beam and to acquire scan data;
an anchor configured to move between an expanded state and a collapsed state, and wherein in said expanded state said anchor anchors said elongated body to walls of said body cavity;
an actuator:
a control device in communication with said transvaginal ultrasound probe, comprising a memory and a control circuitry, wherein said control circuitry controls the actuator to move the transvaginal ultrasound probe and signals said at least one ultrasound transducer to generate said ultrasound beam when said anchor is in said expanded state.

30. A system according to claim 29, wherein said anchor surrounds at least partially said at least one ultrasound transducer; and/or wherein said anchor comprises a balloon, and wherein in an expanded state said balloon is filled with a fluid that allows passage of said ultrasound beam towards a wall of said body cavity in contact with said balloon.

31-47. (canceled)
